(19) United States
(12) Patent Application Publication
(10) Pub. No.: US 2006/0003338 A1
(43) Pub. Date: Jan. 5, 2006

(54) System and methods for the management and treatment of vascular graft disease

(76) Inventors: David Xing-Fei Deng, Mountain View, CA (US); Anya Tsalenko, Chicago, IL (US)

Correspondence Address: AGILENT TECHNOLOGIES, INC.;Legal Department, DL429, Intellectual Property Administration, P.O. Box 7599, Loveland, CO, 80537-0599, US

(21) Appl. No.: 10/882,014

(22) Filed: Jun. 30, 2004

(51) Int. Cl. C12Q 1/68; G01N 33/53; C12M 1/34

(52) U.S. Cl. 435/6; 435/7.1; 435/287.2

(57) ABSTRACT

Various embodiments of the present invention are directed to a system and methods for minimizing risk factors that contribute to the development of vascular graft disease and vascular graft failure. In one embodiment, a microarray-based gene expression analysis may be employed to select pre-implanted vessel candidates suitable as grafts in various vascular transplantation procedures. By using a microarray that includes a set of probe sequences that statistically correlate with vascular graft disease, mRNA expression levels of vascular-graft-disease-related genes within vessel-graft candidates can be determined to produce an expression profile for each vessel tested. Such molecular profile of genes related to various forms of vascular graft disease enables clinicians to select a vessel graft having the lowest probability of developing vascular graft diseases, and having the highest probability of maintaining adequate patency rate. Various types of arteries and veins may be discriminated from one another based on their respective gene expression profiles.

### BACKGROUND OF THE INVENTION

The present invention relates to profiling gene expression levels within vascular tissues.

One embodiment of the present invention provides a system and methods for selecting a vascular tissue graft for various types of vascular transplantation procedures. In order to facilitate discussion of the present invention, a general background for microarrays is provided, below. In the following discussion, the terms “microarray,” “molecular array,” and “array” are used interchangeably. The terms “microarray” and “molecular array” are well known and well understood in the scientific community. As discussed below, a microarray is a precisely manufactured tool which may be used in research, diagnostic testing, or various other analytical techniques.

Array technologies have gained prominence in biological research and are likely to become important and widely used diagnostic tools in the healthcare industry. Currently, microarray techniques are most often used to determine the concentrations of particular nucleic-acid polymers in complex sample solutions. Molecular-array-based analytical techniques are not, however, restricted to analysis of nucleic acid solutions, but may be employed to analyze complex solutions of DNA, RNA, or proteins that can be optically or radiometrically scanned and that can bind with high specificity to complementary molecules synthesized within, or bound to, discrete features on the surface of an array. Because arrays are widely used for analysis of nucleic acid samples, the following background information on arrays is introduced in the context of analysis of nucleic acid solutions. However, the analysis of gene expression levels is not limited to microarrays, but may include any known methods for ascertaining gene expression such as Real-Time PCR, protein array, and proteomics.

Double-stranded DNA may be denatured, or converted into single stranded DNA, by changing the ionic strength of the solution containing the double-stranded DNA or by raising the temperature of the solution. Single-stranded DNA polymers may be renatured, or converted back into DNA duplexes, by reversing the denaturing conditions, for example by lowering the temperature of the solution containing complementary single-stranded DNA polymers. During renaturing or hybridization, complementary base pairs of AT and GC, also known as Watson-Crick (“WC”) base pairs, within anti-parallel DNA strands form in a cooperative fashion, leading to reannealing of the DNA duplex. Strictly A-T and G-C complementarity between anti-parallel polymers leads to the greatest thermodynamic stability, but partial complementarity including non-WC base pairing may also occur to produce relatively stable associations between partially-complementary polymers. In general, the longer the regions of consecutive WC base pairing between two nucleic acid polymers, the greater the stability of hybridization between the two polymers under renaturing conditions.

The ability to denature and renature double-stranded DNA has led to the development of many extremely powerful and discriminating assay technologies for identifying the presence of DNA and RNA polymers having particular base sequences or containing particular base subsequences within complex mixtures of different nucleic acid polymers, other biopolymers, and inorganic and organic chemical compounds. One such methodology is the array-based hybridization assay. FIGS. 1-4 illustrate the principle of the array-based hybridization assay. An array (102 in FIG. 1) comprises a substrate upon which a regular pattern of features is prepared by various manufacturing processes. The array 102 in FIG. 1, and in subsequent FIGS. 2-4, has a grid-like 2-dimensional pattern of square features, such as feature 104 shown in the upper left-hand corner of the array. Each feature of the array contains a large number of identical oligonucleotides covalently bound to the surface of the feature. These bound oligonucleotides are known as probes. In general, chemically distinct probes are bound to the different features of an array, so that each feature corresponds to a particular nucleotide sequence. In FIGS. 1-3, the principle of array-based hybridization assays is illustrated with respect to the single feature 104 to which a number of identical probes 105-109 are bound. In practice, each feature of the array contains a high density of such probes but, for the sake of clarity, only a subset of these are shown in FIGS. 1-3.

Once an array has been prepared, the array may be exposed to a sample solution of target DNA or RNA molecules (110-113 in FIG. 1) labeled with fluorophores, chemiluminescent compounds, or radioactive atoms 115-118. For example, the sample RNA can be directly labeled or labeled after RNA amplification. Labeled target DNA or RNA hybridizes through base-pairing interactions to the complementary probe DNA, which can be deposited or synthesized on the surface of the array. FIG. 2 shows a number of such target molecules 202-204 hybridized to complementary probes 205-207, which are in turn bound to the surface of the array 200. Targets, such as labeled DNA or RNA molecules 208 and 209, that do not contains nucleotide sequences complementary to any of the probes bound to array surface do not hybridize to generate stable duplexes and, as a result, tend to remain in solution. The sample solution is then rinsed from the surface of the array, washing away any unbound-labeled DNA or RNA molecules. In other embodiments, unlabeled target sample is allowed to hybridize with the array first. Typically, such a target sample has been modified with a chemical moiety that will react with a second chemical moiety in subsequent steps. Then, either before or after a wash step, a solution containing the second chemical moiety bound to a label is reacted with the target on the array. After washing, the array is ready for scanning. Biotin and avidin represent an example of a pair of chemical moieties that can be utilized for such steps.

Finally, as shown in FIG. 3, the bound labeled DNA molecules are detected via optical or radiometric scanning. Optical scanning involves exciting labels of bound labeled DNA molecules with electromagnetic radiation of appropriate frequency and detecting fluorescent emissions from the labels, or detecting light emitted from chemiluminescent labels. When radioisotope labels are employed, radiometric scanning can be used to detect the signal emitted from the hybridized features. Additional types of signals are also possible, including electrical signals generated by electrical properties of bound target molecules, magnetic properties of bound target molecules, and other such physical properties of bound target molecules that can produce a detectable signal. Optical, radiometric, or other types of scanning produce an analog or digital representation of the array as shown in FIG. 4, with features to which labeled target molecules are hybridized similar to 406 optically or digitally differentiated from those features to which no labeled DNA molecules are bound. In other words, the analog or digital representation of a scanned array displays positive signals for features to which labeled DNA molecules are hybridized and displays negative features to which no, or an undetectably small number of, labeled DNA molecules are bound. Features displaying positive signals in the analog or digital representation indicate the presence of DNA molecules with complementary nucleotide sequences in the original sample solution. Moreover, the signal intensity produced by a feature is generally related to the amount of labeled DNA bound to the feature, in turn related to the concentration, in the sample to which the array was exposed, of labeled DNA complementary to the oligonucleotide within the feature.

### SUMMARY OF THE INVENTION

Various embodiments of the present invention are directed to a system and methods for minimizing risk factors that contribute to the development of vascular graft disease and vascular graft failure. In one embodiment, mRNA and/or protein expression analyses by various methods including microarray, real-time PCR, and protein array, may be employed to select pre-implanted vessel candidates suitable as grafts in various vascular transplantation procedures. By using a set of probe sequences that statistically correlate with vascular graft disease, a set of various mRNAs and/or protein expression levels of vascular-graft-disease-related genes within vessel-graft candidates can be determined so that an expression profile for each vessel tested can be produced using the determined set. A molecular profile of genes/proteins related to various forms of vascular graft disease enables clinicians to choose the best therapeutic approach for an individual patient from among a number of options, including bypass surgery, stenting, and angioplasty. In addition, a molecular profile of genes/proteins enables clinicians to select a suitable vessel graft having the lowest probability of developing vascular graft diseases, and having the highest probability of maintaining adequate patency rate. Various types of arteries and veins may be discriminated from one another based on their respective gene/protein expression profiles.

In the described embodiments, examples of probe molecules containing gene sequences related to various forms of vascular graft disease that are useful as genetic markers have been identified. A set of genetic markers for vascular graft disease includes genes/proteins that may correlate with the development and progression of thrombosis, intimal hyperplasia, and graft arteriosclerosis, which are clinically recognized, progressive pathological states associated with the development of vascular graft disease. Gene expression profiles of individual patients enable individualized diagnostic, prognostic, and therapeutic care for patients undergoing various types of vascular graft transplantation.

### BRIEF DESCRIPTION OF THE DRAWINGS

FIG. 1-4 illustrate principles of the microarray-based hybridization assay.

FIG. 5 is a generalized diagram showing an exemplary coronary bypass which may be performed using a vein graft or an artery graft for treating a patient with coronary artery disease.

FIG. 6 is an illustration of known mediators of early thrombosis, a pathological state which develops after the first month of surgery.

FIGS. 7A-B are illustrations of morphological changes that occur within a vessel wall associated with progressive intimal hyperplasia, a pathological state that develops between the first month and first year after surgery.

FIGS. 8A-B illustrate differences between different forms of arteriosclerosis, including arteriosclerosis that develops in native vessels and arteriosclerosis that develops in vascular grafts.

FIG. 9 is a generalized representation of a microarray comprising probe molecules with gene sequences determined to be related to vascular graft disease or vascular graft failure.

FIG. 10A illustrates various sets of probe sequences included in a hypothetical microarray substrate for evaluating different vessel biopsies.

FIG. 10B illustrates the results of a hypothetical experiment showing relative signal intensities for a gene expression pattern indicating healthy tissue.

FIG. 10C illustrates the results of a hypothetical experiment showing relative signal intensities for a gene expression pattern indicating a tissue that is at risk for developing a form of vascular graft disease.

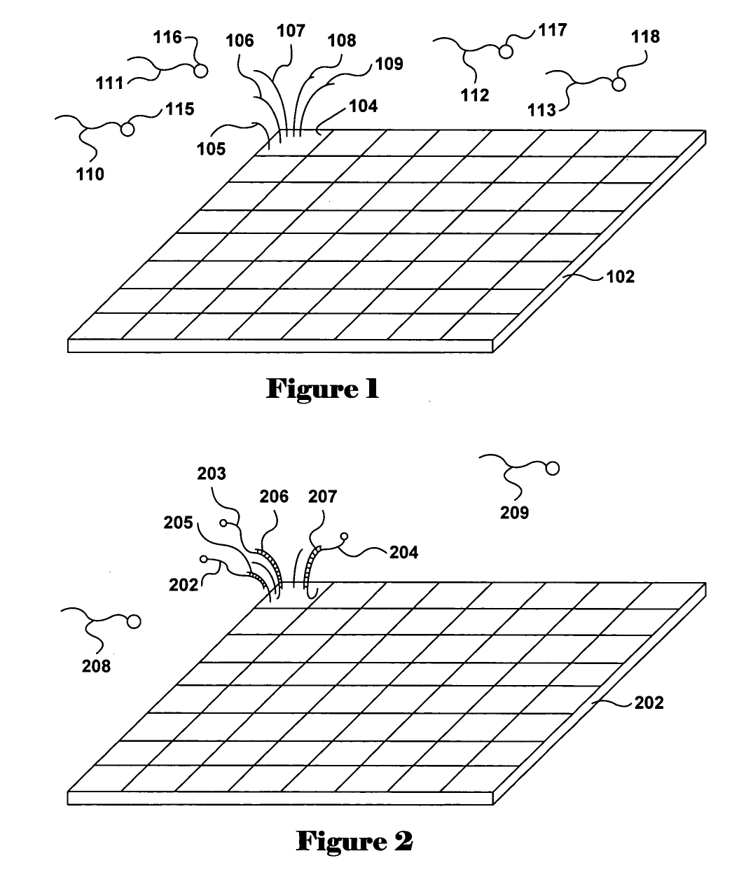

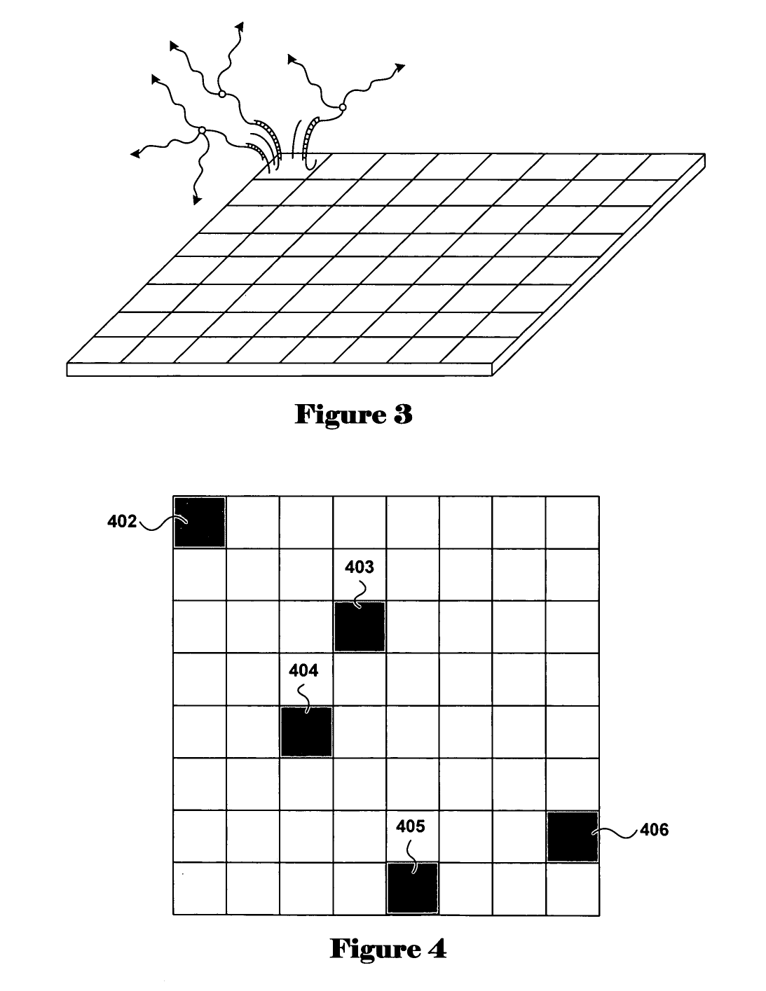

### DETAILED DESCRIPTION OF THE INVENTION

Embodiments of the present invention are generally directed to a system and methods for determining the expression levels of a set of genes that correlate statistically with pathological states associated with vascular graft disease. Vascular graft disease is a clinical condition in which transplanted vessel tissue and surrounding vasculatures undergo morphological and cellular changes as a result of vessel transplantation. Clinical manifestations of vascular graft disease include partial or complete occlusion of the affected vasculatures that arise from the development of various pathological states associated with vascular graft disease, including early thrombosis, intimal hyperplasia, and graft arteriosclerosis. In general, these pathological states or stages related to the development and/or progression of vascular graft disease result in lower patency rates, or reduced rates of blood-flow, through the affected vessels.

To facilitate the present disclosure, a discussion of various forms of vascular reconstructive transplantations and the clinical manifestations associated with various forms of vascular graft disease are provided below.

### Development of Vascular Graft Disease Pursuant to Vascular Transplantation

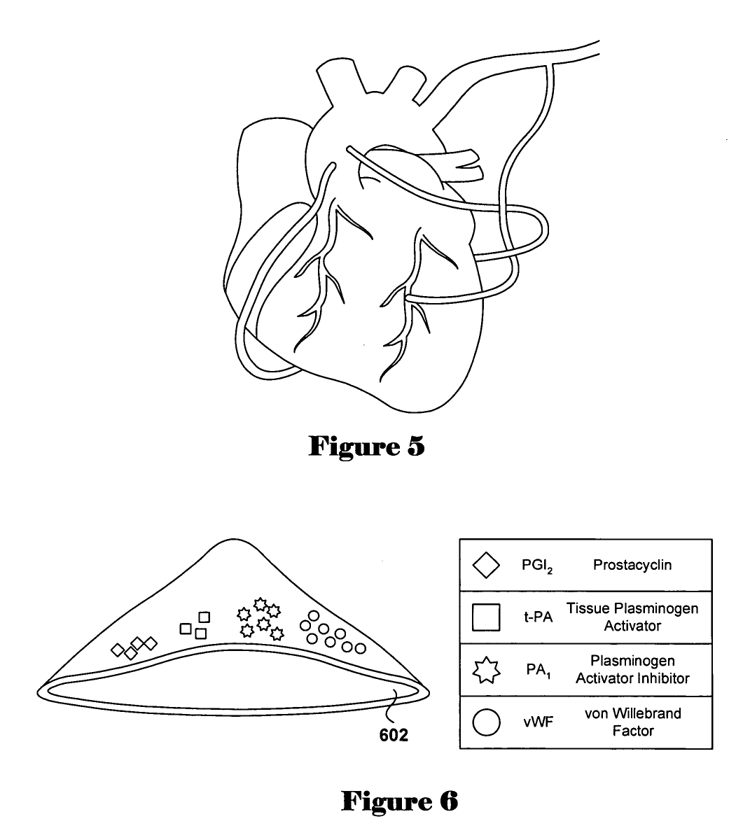

Coronary artery disease is one of a multitude of vascular dysfunctions that globally strikes hundreds of thousands of new victims each year. For patients afflicted with coronary artery diseases, vascular reconstructive surgery, including coronary artery angioplasty and bypass graft surgery, may be necessary in order to improve circulation into tissue areas that receive a reduced blood-flow supply through diseased, native vessels. Different types of grafts used for vascular reconstruction include: (1) non-diseased portions of an autogenous, vascular tissue removed from a diseased patient, (2) prosthetic conduits made of synthetic materials, and (3) composite conduits consisting of prosthetics and vascular tissue. FIG. 5 is a generalized diagram showing an exemplary coronary bypass that may be performed using a vein graft or an artery graft for treating a patient with coronary artery disease. Such grafts are placed into sites of diseased vasculatures in order to replace occluded portions of diseased vessels and to provide alternative conduits for blood flow. In transplanting autogenous tissue, portions of non-diseased arteries and veins are identified within a diseased patient, removed from a non-diseased, native vasculature site within the patient, and re-implanted into a diseased vasculature site within the same patient. Surgeons commonly prefer autogenous conduits over synthetic conduits, particularly for use in reconstructing an infected operative field, or a secondary mesenteric prosthetic bypass graft exhibiting thrombotic complications (Modrall et al., J. Vasc. Surg., 37: No. 2, 2003).

Starting in the mid-1960's, cardiologists experimented with peripheral vessels, such as “saphenous veins,” and increasingly advocated the use of saphenous vein grafts for treating various forms of cardiovascular dysfunctions. Prior to surgery, the saphenous veins of a diseased patient are inspected in situ by identifying healthy vessels having relatively large diameters for circulating adequate volumes of blood. Imaging technologies, such as angiography, computed tomography, and ultrasonography, may be employed to visually inspect vessel candidates prior to graft harvesting. Although saphenous vein grafting provides substantial relief for many patients, and substantially improves the long-term prognosis for certain patient subgroups, the clinical efficacy of vein grafts is relatively short-lived. Within the first year after surgery, approximately 12-20% of saphenous vein grafts fail, due mainly to a phenomenon called “occlusive thrombosis” that leads to progressively diminished rates of blood-flow, a condition referred to as a “lower patency rate.” Within the first month after bypass surgery, thrombosis develops in up to 10-15% of saphenous vein grafts, with or without initial symptoms. Typically, within ten years after surgery, only 60% of saphenous vein grafts remain open, and only 50% of these patent vein grafts maintain high patency rates. Clinicians have long recognized that most vascular graft substitutes are not as functionally competent as the native, non-diseased vessel conduit in maintaining comparable blood-flow rates.

For the treatment of vascular graft disease, which includes pathological states such as early thrombosis, intimal hyperplasia, and graft arteriosclerosis, clinicians often recommend multiple vascular surgeries in order to alleviate the symptoms arising from occluded grafts and to manage the progression of the native disease. For example, multiple numbers of bypasses, reimplantations, thrombectomy, and patch angioplasty procedures may be necessary in order to increase the cumulative patency rate of blood-flow to the engrafted tissue area. The total number of transplantations within an affected tissue area can affect the cumulative patency rate within the area. In addition, some studies suggest that the sequence of graft tissue types used may also affect the ultimate fate of a graft. Improved final patency rates can be achieved if the first bypass is performed using autogenous vein, in contrast to using composite prosthetic vein bypasses that may result in low 2-year patency rates of 12% to 37% (Henke et al., J. Vasc. Surg., 35: 902-9, 2002). Patients with failed prosthetic polytetrafluoroethylene (“PTFE”) bypasses are more likely to develop limb-threatening ischemia that may lead to limb amputations. In contrast, failed autogenous, vein grafts are more amenable to undergoing repeated reconstructions and avoiding needless amputations.

In addition to the increasing frequency of coronary bypasses performed each year, the rate of lower extremity, peripheral-vascular-arterial occlusive diseases appears to be increasing, and an increasing need for operative bypass procedures is therefore expected. For most primary autogenous vein “infrainguinal bypass” reconstructions, a 5-year patency term is expected (Henke et al., J. Vasc. Surg., 35: 902-9, 2002). Limb salvage rates for infrainguinal bypasses are between 70% and 85%, independent of whether the vein bypass is reversed in situ, or in a non-reversed, translocated configurations. In general, prosthetic bypasses demonstrate lower patency and limb salvage rates when compared with autogenous vein grafts, but they may be suitable for above-knee-femoral-popliteal-artery-bypass procedures. Bypass patency rates for secondary infrainguinal bypasses are substantially lower than for primary bypasses, which may be attributed to multiple factors, including the advanced stage in the progression of the arterial disease, reduced availability of autogenous conduits and re-operative fields, and lack of optimal arterial tissue candidates.

For the treatment of arteriosclerosis associated with ischemic foot complications in patients with diabetes mellitus, the dorsalis-pedis-artery bypass (“DP”) is a commonly performed arterial reconstruction for the treatment of diseased extremities. Typically, a comprehensive, intra-arterial, digital subtraction angiography is performed in order to obtain an image of the circulation from the renal arteries to the base of the toes of patients so that the quality of the DP artery and the potential use of the DP artery as an outflow artery can be determined. The decision to perform a DP bypass is based on anatomic and clinical factors (Pomposelli et al., J. Vasc. Surg., 37: 307-15, 2003). For example, a DP artery may be chosen as an outflow vessel candidate when alternative outflow artery is not proximately located, or when the DP artery is determined by an arteriogram to represent the healthiest and largest vessel candidate. However, if a femoral-popliteal bypass or a tibial-arterial bypass can restore a palpable foot pulse, and tissue loss is one indicator of low patency rate, a DP bypass may not be performed. In cases in which patients are suffering from tissue loss or gangrene, the DP bypass may be preferentially selected instead of a patent, peroneal artery of the patient. However, if the DP artery is a poorer quality vessel as determined by an arteriogram, or when the vein candidate is inadequate in length for reaching the DP artery, a peroneal-artery bypass may be performed. In selecting potential graft tissue to be used for bypass procedures, the simplest and most expedient approach is to make open incisions in order to identify saphenous vein candidates for use as grafts, while eliminating vessels that are diseased or narrowed, or that display other properties that negate optimal patency rates. In the absence of adequate saphenous veins that can be harvested from the legs of a patient, available arm veins may be alternatively used. When competent arm veins are not available, then less than optimal saphenous vein may be used, provided that the caliber and quality of the saphenous vein is adequate for bypass procedures.

As an alternative to using saphenous veins in cardiac bypass surgery, arterial conduits, such as chest artery, have been engrafted into affected areas, resulting in improved engrafted patency rates that are higher than patency rates associated with saphenous vein conduits. Despite this trend, the autogenous saphenous vein is a graft of choice for more than 70% of all bypass grafts, and saphenous vein remains as a strong choice for patients who depend on multiple vessel graft transplantations for the preservation of a life or a limb. Currently available procedures and related technologies, some of which have been described above, for inspecting vessel tissue as graft candidates and for treating occluded vessel grafts are limited to low resolution imaging techniques and conventional surgical methods. A need for improved technologies and more sensitive methods for the management of risk factors associated with the development of vascular graft disease has been recognized by cardiologists and general clinicians.

### Embodiments of the Present Invention

The expression levels of vascular-graft-disease-related genes can be determined for pre-implanted vascular graft tissues and for post-implanted vascular grafts in order to assess patient risk factors for developing various forms of vascular graft disease, with various pathophysiological changes, such as early thrombosis, intimal hyperplasia, graft arteriosclerosis, and the like. The embodiments of the present invention can be employed for the prevention, management, and treatment of vascular graft disease in patients who are candidates for vascular surgery and/or who have already undergone vascular surgery. The described embodiments may be employed before and/or after a variety of transplantation procedures, including coronary artery angioplasty and coronary bypass graft surgeries, reconstructions of ischemic or gangrenous bowel, surgeries to alleviate secondary mesenteric prosthetic bypass exhibiting thrombotic complications, bypass procedures for the treatment of lower extremity peripheral-vascular-arterial occlusive diseases, such as infrainguinal bypasses and above-knee-femoral-popliteal-artery bypasses. Other embodiments are directed to the prevention, management, and treatment of vascular graft disease that may arise from surgeries for the treatment of ischemic foot complications in patients with diabetes mellitus, including dorsalis-pedis-artery bypasses, femoral-popliteal bypasses, tibial-arterial bypasses, and peroneal-artery bypasses, for example. The described embodiments relate to transplantation procedures involving autogenous tissue as grafts, including various types of non-diseased arteries and veins, such as saphenous veins, that can be identified within a diseased patient, removed from a non-diseased, native vasculature site within the patient, and re-implanted into a diseased vasculature site within the same patient.

The general phenomenon of vascular graft disease often develops as a secondary clinical manifestation after a reconstructive vascular surgery performed to treat diseased, native vasculatures. Affected patients must undergo surgical remedial procedures, such as angioplasty or bypass surgery, in order to correct for partial or complete occlusion caused by the native condition. Due to biochemical and mechanical trauma introduced by graft transplantation, vascular graft disease may develop within the engrafted vessel when the vessel graft is an autogenous tissue, or within native vasculatures in situ surrounding the engrafted vessel when the vessel graft is a prosthetic material. Vasculatures that develop vascular graft disease exhibit morphological changes within the affected vessels that may arise from changes in the cellular composition of the diseased vessel. Changes at the cellular level may result from the de novo production of structural proteins and soluble protein factors that play a role in activating various biochemical mechanisms, which can be induced by graft transplantation. For transplantations using autogenous vessel grafts, the re-implantation of a non-diseased vessel removed from a native tissue site into a non-native vasculature site may undergo biochemical changes that can lead to the development of various pathological states associated with vascular graft disease.

Generally, any form of vascular graft disease results in reduced blood-flow rates or lower patency rates due to the post-operative development of occlusions within the affected vessel. This diminished capacity to transfer blood at flow rates comparable to non-diseased vessel is used to gauge the flow capacity of the vessel graft. Low patency rates correlate with pathological states associated with vascular graft disease and may result in vascular graft failure due to the formation of a thrombotic occlusion within the affected vessel. FIGS. 6-8 illustrate three distinguishable pathological states that involve sequential, interrelated pathological processes. FIG. 6 is an illustration of known mediators of early thrombosis, a pathological state that develops after the first month of surgery. Early thrombosis is a major cause of graft occlusion that involves the activation of pro-coagulent mechanisms mediated by surface vascular endothelial cells in response to biochemical and mechanical injuries resulting from traumas associated with graft explantation and re-implantation. In FIG. 6, a cross-section of a hypothetical vessel 602 comprising vascular endothelial cells is shown. Anticoagulent mechanisms are mediated by prostacyclin (“PGI₂”), thrombomodulin, tissue plasminogen activator (“t-PA”), and heparin sulfates. Known procoagulant mediators include the plasminogen activator inhibitor (“PAI”) and von Willebrand factor (“vWf”).

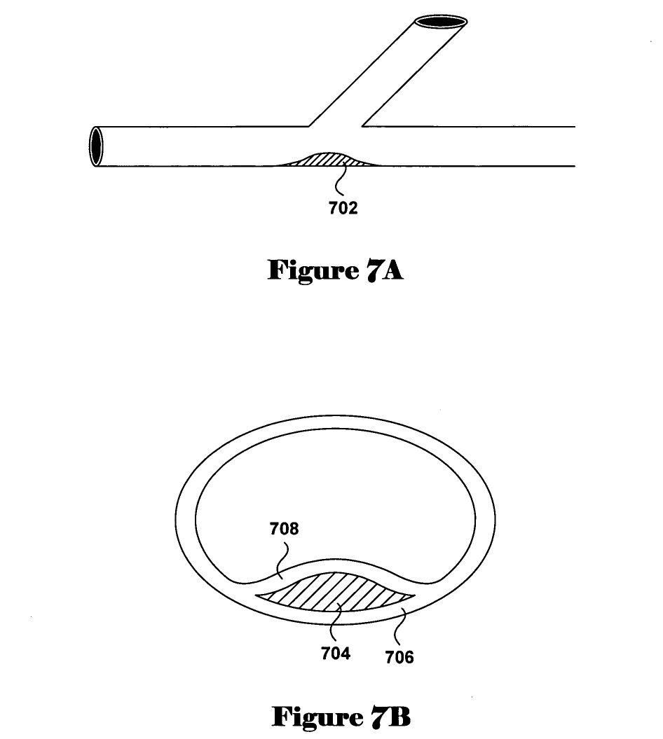

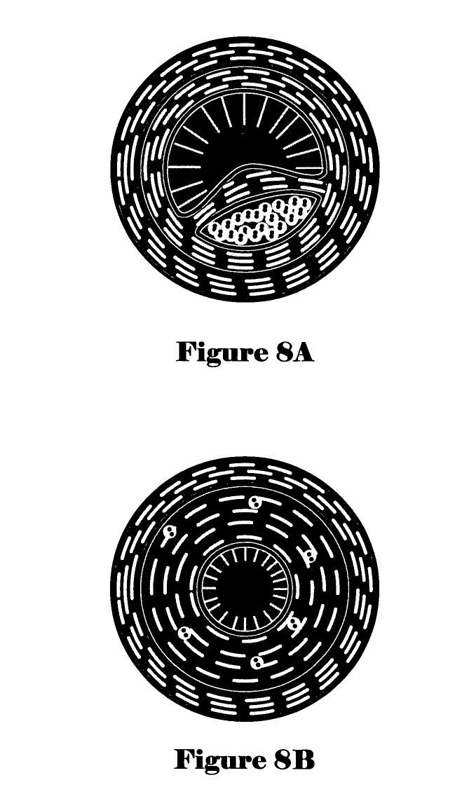

FIGS. 7A-B illustrate morphological changes that occur within a vessel wall associated with progressive intimal hyperplasia, a pathological state that develops between the first month and first year after surgery. The progressive intimal hyperplasia involves endothelial cell activation, fibroblast and inflammatory cell recruitment, release of cytokines and growth factors that stimulate vascular smooth muscle cell migration and proliferation, and deposition of newly formed, extracellular matrix and production of extracellular matrix proteins. FIG. 7A shows a longitudinal representation of a hypothetical intimal layer 702 of a vessel wall. FIG. 7B shows a cross-sectional view of the intimal layer 704, in which cellular proliferation that occurs between the endothelial cell layer 706 and smooth muscle cell layer 708 contributes substantially to reducing the patency rate and to supporting the structural foundation for the development of graft atheroma or graft arteriosclerosis at a later time. Intimal hyperplasia develops in vein grafts during endothelial cell regeneration process, whereas intimal hyperplasia of arterial grafts occur during endothelial cell denudation. FIGS. 8A-B illustrate differences between two different forms of arteriosclerosis: (1) arteriosclerosis that develops in native vessels; and (2) arteriosclerosis that develops in vascular grafts. The degeneration of vessel grafts is progressive. Graft-specific arteriosclerosis generally develops after the first year of transplantation. Indicators of graft-specific arteriosclerosis include concentric lesion formation, absence of lipid core deposition, and diffuse narrowing of the engrafted or implanted vessel. The development of these vascular pathological states involves partial or complete occlusion of the grafted vessels resulting in reduced patency rates.

The system and methods of the present invention can be employed by clinicians for managing the treatment of patients who have a need for vascular reconstructive surgery to correct vascular dysfunctions affecting native vessels. For example, the present method may be used to facilitate the treatment of patients who are candidates for primary bypass surgery suffering from various forms of coronary artery diseases, or to facilitate the treatment of various forms of occlusive diseases that affects peripheral vascular structures, as is common for patients with Diabetes Mellitus. The present invention can be applied to vascular graft samples that can be removed from a patient who may be at risk for developing a form of vascular graft disease. In one embodiment, the biopsy sample may represent pre-implanted, candidate graft tissue that is evaluated for gene expression patterns in order to distinguish healthy cells from unhealthy cells during the graft-selection process. Removal of multiple tissue samples from a single patient is suitable for some patient sub-populations, for example, for patients who have not undergone multiple numbers of bypasses. Biopsies removed before reconstructive surgery can be employed in determining gene expression patterns for a number of reasons including: (1) to identify the abnormal expression levels of genes that may indicate higher risks for developing vascular graft disease, (2) to estimate the risks of patients for developing complications of vascular graft disease, (3) to predict the long-term patency rate for a given vessel candidate, and (4) to correct abnormal gene expression within candidate vessel prior to bypass surgery via gene therapy and/or pharmacological agents.

In another embodiment, the biopsy sample may be obtained from any portion of an implanted vessel tissue that remains after vascular surgery, which can be used by clinicians to formulate modes of therapy based on the gene-expression-pattern information derived from a graft sample. Usage of remnant tissue for gene expression evaluation may be suitable, for example, in patients who cannot provide multiple numbers of biopsies for various reasons, including the unavailability of multiple numbers of graft vessel candidates. The gene-expression-pattern information derived from remnants of pre-implanted vessels can be used to estimate or to predict the severity of complications or long-term effects arising from the vascular transplantation. The determination of gene expression patterns for each patient can be used by physicians to provide patient-specific mode of therapy. For example, a low level of thrombomodulin may indicate high risk of thrombosis development in the early post-surgery stage.

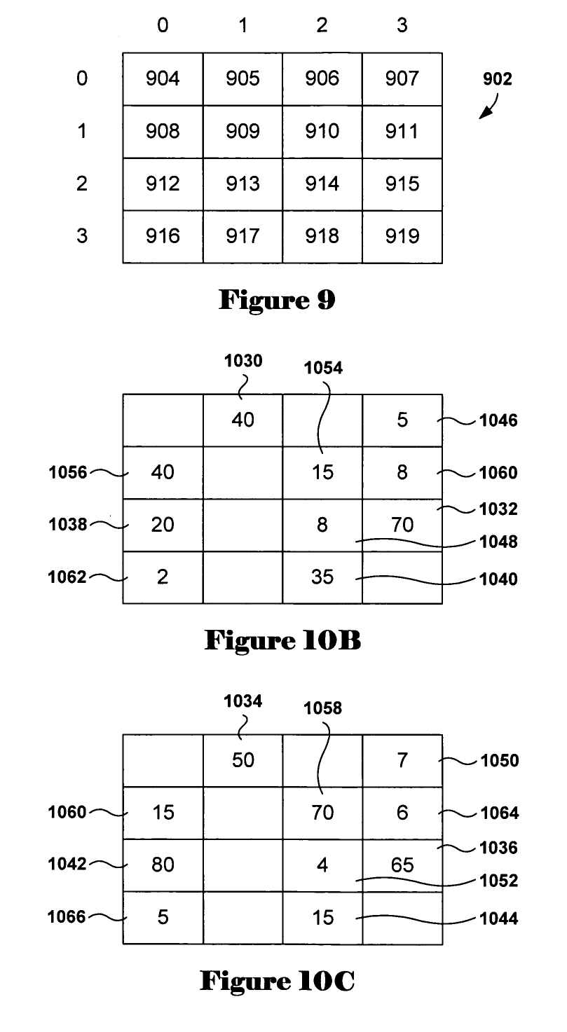

The embodiments of the present invention include various methods such as real-time PCR (e.g., Taqman®) for measuring mRNA levels, proteomics techniques for measuring protein levels, protein arrays for measuring protein levels, and DNA arrays for measuring mRNA levels. For these methods and other equivalent methods, a set of biomarkers (genes and/or proteins) disclosed by the present invention can be used to predict the risks for developing vascular graft disease. In the following figures and descriptions of embodiments, the embodiments will be described using a microarray as one exemplary method. FIG. 9 is a generalized representation of a microarray that includes probe molecules with gene sequences determined to statistically correlate with the development of vascular graft disease or vascular graft failure. Probe molecules, for example, may target mRNA fragments or cDNA copies of mRNA fragments of biomarker genes. Exact complementarity between probes and target fragments is not necessary. Instead, the probes need only be sufficiently complementary in order to stably hybridize with the intended target fragments. In FIG. 9, a hypothetical microarray with features arranged in a small, 4×4 array 902 is shown for simplicity. The microarray can include multiple sets of probes, including sets that represent either control probes or vascular-graft-disease-related probes. For example, some probe sets may include: (1) probe sequences determined to be expressed in only vascular cell types 904-907 that can be derived from veins and arteries as one form of positive control, (2) probe sequences determined not to be expressed in vascular cell types 908-911, as one form of negative control, (3) probe sequences determined to statistically correlate with vascular graft disease 912-915, and (4) probe sequences determined to not correlate statistically with vascular graft disease 916-919. Although vascular tissue is composed of a mixture of multiple cell types, in general, vascular tissue is predominantly comprised of variable ratios of endothelial cells and smooth muscle cells. A microarray containing one or more of these four possible subsets of probe molecules can be used to determine a gene expression pattern for a given biopsy tissue from a particular patient in a high throughput manner. Probe molecules, such as probes 912-915, are determined to statistically correlate with vascular graft disease when the hybridization of vessel-graft-derived biological samples to such probe sequences reasonably correlate with clinically-recognized, pathological states associated with various forms of vascular graft disease and pathophysiological states (early thrombosis, intimal hyperplasia, graft athelerosclerosis, and various related processes). Various gene expression patterns may indicate a risk for developing vascular-graft-disease. Each gene expression pattern represents a binding pattern that includes the binding activities of one or more combinations of vascular-graft-disease-related probes. Although a single vascular-graft-disease-related probe may indicate a risk for developing vessel-graft-disease, an expression pattern comprising a combination of vascular-graft-disease-related genes may provide more reliable data for making clinical decisions.

Probe sequences may comprise single-stranded or double-stranded forms of genomic DNA, cDNA, oligonucleotides, RNA, including any chemical modifications of these polynucleotide forms. Array-based assays can involve other types of biopolymers, synthetic polymers, and other types of chemical entities. A biopolymer is a polymer of one or more types of repeating units. Biopolymers are typically found in biological systems and particularly include polysaccharides, peptides, and polynucleotides, as well as their analogs such as those compounds composed of, or containing, amino acid analogs or non-amino-acid groups, or nucleotide analogs or non-nucleotide groups. This includes polynucleotides in which the conventional backbone has been replaced with a non-naturally occurring or synthetic backbone, and nucleic acids, or synthetic or naturally occurring nucleic-acid analogs, in which one or more of the conventional bases has been replaced with a natural or synthetic group capable of participating in Watson-Crick-type hydrogen bonding interactions. Polynucleotides include single or multiple-stranded configurations, where one or more of the strands may or may not be completely aligned with another. For example, a biopolymer includes DNA, RNA, oligonucleotides, and PNA and other polynucleotides as described in U.S. Pat. No. 5,948,902 and references cited therein, regardless of the source. An oligonucleotide is a nucleotide multimer of about 10 to 100 nucleotides in length, while a polynucleotide includes a nucleotide multimer having any number of nucleotides.

As an example of a non-nucleic-acid-based microarray, protein antibodies may be attached to features of the array that would bind to soluble labeled antigens in a sample solution. Many other types of chemical assays may be facilitated by array technologies. For example, polysaccharides, glycoproteins, synthetic copolymers, including block copolymers, biopolymer-like polymers with synthetic or derivitized monomers or monomer linkages, and many other types of chemical or biochemical entities may serve as probe and target molecules for array-based analysis. A fundamental principle upon which arrays are based is that of specific recognition, by probe molecules affixed to the array, of target molecules, whether by sequence-mediated binding affinities, binding affinities based on conformational or topological properties of probe and target molecules, or binding affinities based on spatial distribution of electrical charge on the surfaces of target and probe molecules.

A set of biomarker genes has been identified using methods described in Example 1, provided in the following subsection. The set of biomarkers consist of at least two genes, at least 5 genes, at least 20 genes, at least 100, at least 500, at least 1,000 genes, at least 2,000, at least 5,000, or at least 10,000 genes or more. One or more of the following list of genes, which includes vascular-graft-disease-related genes, can be used to represent probe molecules 912-915 as described previously in FIG. 9. The Appendix section, incorporated herein by reference, provides a list of genes grouped here, according to various known functions and representing examples of biomarker-genes associated with various pathophysiological changes of vascular graft diseases, including genes related to: (1) cell proliferation and migration; (2) inflammation and immune response; (3) coagulation and thromobosis; (4) extracellular matrix and cellular adhesion; (5) transcriptional regulation; (6) signal transduction; and (7) other functions. Further descriptions of data incorporated into the Appendix are provided in Example 1 provided below.

Microarrays containing vascular-graft-disease-related gene probes can be fabricated using drop deposition from pulsejets of either polynucleotide precursor units (such as monomers) in the case of in situ fabrication, or the previously obtained polynucleotide. Such methods are described in detail in, for example, U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. Other drop deposition methods can be used for fabrication, as previously described herein. Also, instead of drop deposition methods, photolithographic array fabrication methods may be used. Interfeature areas need not be present particularly when the arrays are made by photolithographic methods as described in those patents.

Microarrays containing vascular-graft-disease-related gene probes can be exposed to a tissue sample including labeled target molecules, or, as mentioned above, to a tissue sample including unlabeled target molecules followed by an exposure to labeled molecules that bind to unlabeled target molecules bound to the array, and the array is then read. Reading of the array may be accomplished by illuminating the array and reading the location and intensity of resulting fluorescence at multiple regions on each feature of the array. For example, a scanner may be used for this purpose, which is similar to the AGILENT MICROARRAY SCANNER manufactured by Agilent Technologies, Palo Alto, Calif. Other suitable apparatus and methods are described in U.S. patent applications Ser. No. 10/087447 “Reading Dry Chemical Arrays Through The Substrate” by Corson et al., and in U.S. Pat. Nos. 6,518,556; 6,486,457; 6,406,849; 6,371,370; 6,355,921; 6,320,196; 6,251,685; and 6,222,664. However, arrays may be read by any other method or apparatus than the foregoing, with other reading methods including other optical techniques, such as detecting chemiluminescent or electroluminescent labels, or electrical techniques, for where each feature is provided with an electrode to detect hybridization at that feature in a manner disclosed in U.S. Pat. No. 6,251,685, and elsewhere.

A result obtained from reading an array may be used in that form or may be further processed so that conclusions can be made based on the gene expression pattern read from the array, such as whether or not a set of vascular-graft-disease-related gene target sequences is present in the tested sample. A gene expression pattern can also indicate whether or not a particular condition of a patient exists, such as whether or not the patient is at risk of developing early thrombosis, intimal hyperplasia, or graft arteriosclerosis. A result of the reading, whether further processed or not, may be forwarded, such as by communication, to a remote location if desired, and received there for further use, such as for further processing. When one item is indicated as being remote from another, this is referenced that the two items are at least in different buildings, and may be at least one mile, ten miles, or at least one hundred miles apart. Communicating information references transmitting the data representing that information as electrical signals over a suitable communication channel, for example, over a private or public network. Forwarding an item refers to any means of getting the item from one location to the next, whether by physically transporting that item or, in the case of data, physically transporting a medium carrying the data or communicating the data.

Scanning of a microarray by an optical scanning device or radiometric scanning device generally produces a scanned image comprising a rectilinear grid of pixels, with each pixel having a corresponding signal intensity. These signal intensities are processed by an array-data-processing program that analyzes data scanned from an array to produce experimental or diagnostic results which are stored in a computer-readable medium, transferred to an intercommunicating entity via electronic signals, printed in a human-readable format, or otherwise made available for further use. Molecular array experiments can indicate precise gene-expression responses of organisms to drugs, other chemical and biological substances, environmental factors, and other effects. Processing of microarray data can produce detailed chemical and biological analyses, disease diagnoses, and other information that can be stored in a computer-readable medium, transferred to an intercommunicating entity via electronic signals, printed in a human-readable format, or otherwise made available for further use.

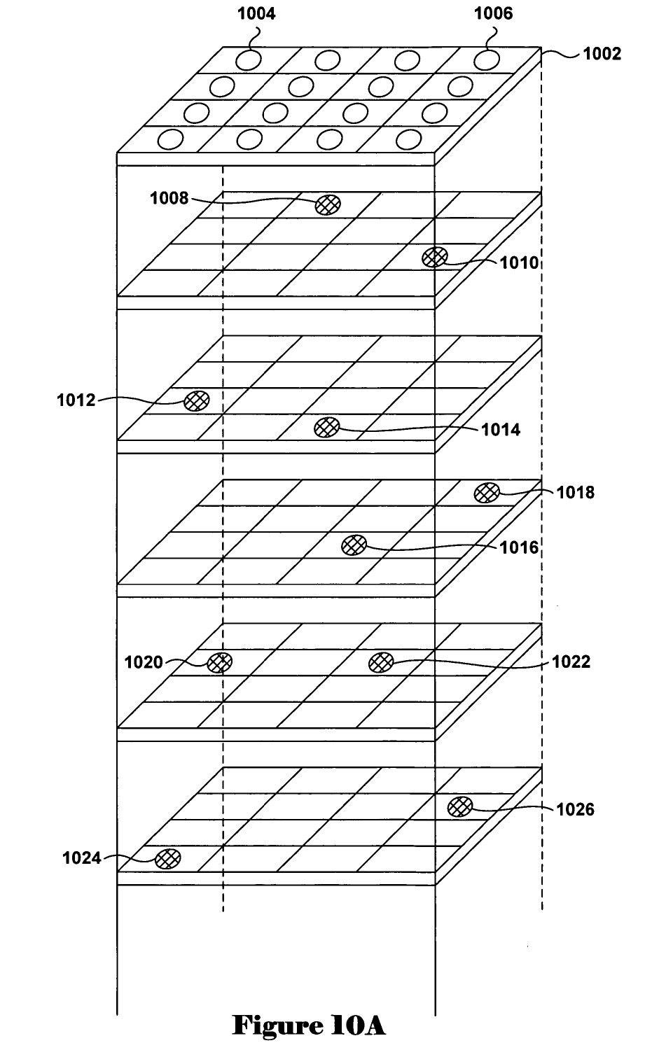

FIG. 10A illustrates the various sets of probe sequences included in a hypothetical microarray substrate for evaluating different vessel biopsies. Vessel samples that are compared may be derived from a single patient or from different patients. Once tissue samples are converted into respective RNA or cDNA solutions, for example, the tissue samples can be labeled differentially and hybridized with a microarray substrate containing various sets of vascular-graft-disease-related probe sequences, including the genes listed in the Appendix, and sets of negative and positive control sequences. For example, in FIG. 10A, a relatively simple 4×4 hypothetical array 1002 containing a set of 16 features containing probe molecules, such as 1004 and 1006, is provided. A 4×4 array is used for simplicity of illustration. Embodiments of the present invention generally include microarrays having hundreds, thousands, tens of thousands, and hundreds of thousands of features. Probe sequences may be selected for various reasons, including sets of probes for distinguishing healthy cells from diseased cells and sets of probes for distinguishing growing cells from necrotic cells. The microarray can include various sets of different types of probe sequences, including, for example: (1) a set of positive control probe molecules, such as probes 1008 and 1010 in FIG. 10A, including quasi-housekeeping genes that are expected to be expressed by most vascular cells in order to maintain a normal or average rate of growth; (2) a set of vascular-graft-disease-related genes, such as probes 1012 and 1014, that are expected to be expressed in endothelial cells preferentially or exclusively; (3) a first negative control set of genes, such as 1016 and 1018, that are not expected to be expressed in most types of endothelial cells; (4) a set of vascular-graft-disease-related genes, such as 1020 and 1022, that may be expressed in smooth muscle cells preferentially or exclusively; and (5) a second negative control set of genes, such as 1024 and 1026, that are not expected to be expressed in most types of smooth muscle cells. Vascular-graft-disease-related genes that are expected to be expressed in endothelial cells may preferentially include both a set of genes that are over-expressed and a set of genes that are under-expressed in unhealthy cells, with respect to healthy cells. Vascular-graft-disease-related genes that are expected to be expressed in smooth muscle cells may preferentially include both a set of genes that are over-expressed and a set of genes that are under-expressed in unhealthy cells, with respect to healthy cells.

FIG. 10B shows hypothetical results of a hypothetical experiment showing relative signal intensities for a gene expression pattern indicating healthy tissue. FIG. 10C shows hypothetical results of a hypothetical experiment showing relative signal intensities for a gene expression pattern indicating a tissue that is at risk for developing a form of vascular graft disease. In FIGS. 10B and 10C, the signal intensity values indicating the binding activity of a selective set of probes described in FIG. 10A are provided. House keeping genes are expected to be expressed in both healthy and unhealthy vascular tissues to a similar degree, as shown by comparing signal intensifies such as 1030 and 1034, and signal intensities 1032 and 1036. Vascular-graft-disease-related genes that are expected to be expressed in endothelial cells preferentially or exclusively can include genes that are over-expressed (compare signal intensifies 1038 and 1042) and genes that are under-expressed (compare signal intensities 1040 and 1044) in diseased cells, with respect to normal cells. One negative control set of genes includes those genes that are not expected to be expressed in most types of endothelial cells (compare signal intensities 1046 and 1050, and signal intensities 1048 and 1052), for example. Vascular-graft-disease-related genes that are expected to be expressed in smooth muscle cells preferentially or exclusively can include genes that are over-expressed (compare signal intensities 1054 and 1058) and genes that are under-expressed (compare signal intensities 1056 and 1060) in diseased cells, with respect to normal cells. Another negative control set of genes includes those genes that are not expected to be expressed in most types of smooth muscle cells (compare signal intensities 1062 and 1066, and 1060 and 1064), for example. The list of genes described in the following subsections, and in the Appendix, can be strategically selected to be incorporated within a microarray design so that various types of diagnostic microarrays can be manufactured.

The example illustrated in FIGS. 10A-C provides a simplified illustration of the data collection and data interpretation involved in microarray-expression analysis. For high-density microarrays, data collection via feature extraction and data interpretation may become increasingly complex, involving sophisticated pattern matching, extensive pattern databases, and various analytical and probabilistic metrics, but microarrays enable a high throughput screening method that provides significant advantages in time and effort. An example of a relatively simple, linear metric is to compute the metric value M by:

$$M = ( {\sum }_{i = 1}^{n} {E}_{i} {w}_{i} ) - ( {\sum }_{i = 1}^{m} {D}_{i} {w}_{i} )$$

where Eᵢ are the observed expression levels of genes expected to exhibit greater expression in healthy tissue than in diseased or potentially diseased tissue, Dᵢ are the observed expression levels of genes expected to exhibit less expression in healthy tissue than in diseased or potentially diseased tissue, and wᵢ are weighting factors for the different observed gene expression levels. The greater the value of M calculated for a tissue, the more likely the tissue will provide a successful graft. More complex, non-linear metrics, in which cross products of observed expression levels for sets of genes are calculated, may more accurately reflect interdependencies between different genes. Alternatively, neural networks may be employed to recognize favorable and unfavorable patterns, or various pattern-matching and searching techniques may be employed to determine which stored patterns most closely resemble observed gene-expression patterns. Multi-channel array experiments may be employed for self-normalization, to concurrently evaluate multiple tissues, or both, using different chemiluminescent, fluorescent, phosphorescent, or radioactive labels to label different samples.

Methods representing embodiments of the present invention that evaluate the health of vessels based on the relative gene expression patterns enable patient-specific treatments that target vascular-graft-disease-specific genes for gene therapy and for treatments with pharmacological agents. Gene-therapy and chemotherapies to prevent or ameliorate graft deterioration can be followed over time, using pre-surgically and post-surgically determined expression levels. Gene expression in candidate graft tissues may be evaluated with respect to similar, non-candidate tissues, to statistical samplings of various tissues obtained from the patient, and to other patient-specific sample sets.

One embodiment comprises various methods for selecting a graft among available vessel candidates. The selected vascular graft should have a low probability of graft failure for use in reconstructive surgery, based on the determination of gene expression patterns by employing a microarray containing vascular-graft-disease-related probe sequences. A set of vascular-graft-disease-related probe sequences includes subsets of gene sequences related to, or specific to, pathological states such as early thrombosis, intimal hyperplasia, and graft arteriosclerosis. A vascular tissue sample from a patient can be removed or explanted as a biopsy prior to vascular transplantation surgery. Suitable tissue samples can be derived from a portion of various autogenous, vascular tissue graft removed from the patient. From the tissue sample, a tissue extract can be derived that comprises a set of target gene sequences in nucleic acid or polypeptide form, which can be labeled by various conventional methods known in the art. Any number of distinct tissue samples can be prepared for simultaneous analysis using a microarray to determine the respective gene expression patterns of each tissue sample tested. A microarray substrate can provide one or more probe molecules that statistically correlate with vascular graft disease, so that clinical decisions can be based on the expression levels of several indicator genes. A tissue sample containing target gene sequences that are complementary to vascular-graft-disease-related probe sequences is recognized by measured signal intensities. One or more reference samples may be prepared and labeled in parallel experiments with the tissue samples of interest. Examples of reference samples include gene sequences that may be isolated from various established cell lines having vascular lineage, various types of autogenous vessels, and various types of non-autogenous vessels. Suitable reference samples will vary depending on the particular clinical context. The relative expression levels for sets of vascular-graft-disease-related genes contained within the various tissue samples tested can be compared. The computed relative expression levels for the various tissue samples can be used for screening pre-implanted vascular tissues among candidate vessels in order to select a vessel suitable as a graft in reconstructive surgery, the selected vessel graft having a low probability of graft failure.

In another embodiment, methods are provided for estimating or predicting the severity of complications or long-term effects arising from a particular vascular transplantation, based on the relative gene-expression patterns for the tested vessel tissue and a reference sample. A vascular tissue sample can be obtained from any portion of an implanted vessel tissue that remains after vascular surgery. Usage of remnant tissue for gene expression evaluation may be suitable, for example, in patients that cannot provide multiple numbers of biopsies for various reasons including the unavailability of multiple numbers of graft vessel candidates. The tissue sample comprises any portion of an autogenous, vascular tissue graft removed from the patient. From the tissue sample, a tissue extract comprising a set of target gene sequences in nucleic acid or polypeptide form can be prepared. A microarray substrate can provide one or more probe molecules that statistically correlate with vascular graft disease, so that clinical decisions can be based on the expression levels of several indicator genes. A set of vascular-graft-disease-related genes includes subsets of gene sequences related, or specific, for pathological states such as early thrombosis, intimal hyperplasia, and graft arteriosclerosis. A reference sample can be prepared from various established cell lines having vascular lineage, various types of autogenous vessel, and various types of non-autogenous vessel. Suitable reference sample will vary depending on the particular clinical context. The relative expression level for a vascular-graft-disease-related gene within the remnant tissue sample can be used to estimate or predict the severity of complications or long-term effects arising form vascular transplantation. The determination of gene expression patterns for each patient can be used by physicians to provide patient-specific mode of therapy, including gene therapy and treatments with pharmacological agents.

In another embodiment, a method for monitoring the progress of a graft and for detecting the development of vascular graft disease is provided. Multiple number of biopsies may be obtained from a patient for analysis at various times in order to monitor the condition of a post-implanted vessel graft, and thus to monitor the risk for developing various forms of vascular graft disease. For example, a first biopsy sample of a pre-implanted vessel can be removed from a patient prior to reconstructive surgery, or can be obtained as a remnant as a left-over vessel tissue during surgery. At a later time, perhaps several months later for example, a second biopsy sample of a post-implanted vascular graft can be removed from a patient. The first and second samples collected at different times can be prepared so that both samples may be simultaneously evaluated using a microarray containing vascular-graft-disease-related probe sequences. The respective gene expression patterns for both samples can be compared in order to monitor changes in gene expression patterns that may indicate the onset of vascular graft disease, which includes early thrombosis, intimal hyperplasia, and graft arteriosclerosis. Comparison of multiple tissue samples removed from a patient at various times is therefore not limited to two samplings.

In another embodiment, a method for enhancing the patency rate of a graft is provided. A vessel graft tissue suitable for transplantation can be removed from a first non-diseased site of a patient. Suitable samples include autogenous, pre-implanted vascular tissue graft removed from a patient or a remnant tissue representing a portion of an implanted vessel removed during surgery. The autogenous tissue assayed includes various artery or vein. The graft tissue can be cultured ex vivo prior to reimplantation into a second diseased site of the patient in need of treatment. The graft tissue can be contacted with a pharmaceutical composition. The pharmaceutical composition may comprise a pharmaceutical carrier and/or a growth factor that stimulates endothelial cells and/or smooth muscle cells. Alternatively, the pharmaceutical composition may comprise a pharmaceutical carrier and/or a plasmid encoding a gene that inhibits the development of vascular graft disease. Alternatively, the pharmaceutical composition may comprise a pharmaceutical carrier and one or more anti-coagulant agents and anti-platelet drugs, with or without plasmids and growth factors described above. The graft tissue can be re-implanted into the second diseased site of the patient. Alternatively, in related embodiments, the graft tissue can be treated with a set of pharmaceutical compositions prior to the re-implantation, and the same or another set of pharmaceutical compositions can be delivered to the patient after the re-implantation in order to continue therapy directed to various forms of vascular graft disease.

For the described embodiments, examples of tissue samples suitable for microarray analysis include major/minor arteries, major/minor veins, capillaries of arteries and veins, and endothelial and smooth muscle cells comprising various arteries and/or veins. Samples also include any solution, mixture, or purified forms of nucleic acids representing genes associated with any mechanism involved in the development of vascular graft disease and related diseases. For example, mRNAs, total cellular RNAs, and cDNA derivatives of mRNAs and total RNAs are contemplated as suitable samples. The target nucleic acid samples may be labeled with any fluorophores, chemiluminescent compounds, or radioactive atoms, by using conventional methods known and practiced by persons skilled in the art. The substrate oligonucleotides may comprise any portion of a gene of interest, and may be represented as single-stranded or double-stranded forms of RNA, cDNA, and genomic DNA, including any modifications of these forms. The oligonucleotides can be attached to the substrate by covalent or noncovalent bonds. The collective display of substrate oligonucleotides on the surface of a microarray may represent a complete genome of an organism, or any subpopulation of genes of particular interest, including particular gene sets associated with the mechanism or phenotype of vascular graft disease. Patterns of overexpression or underexpression of genes that correlate with the development of thrombosis, intimal hyperplasia, and arteriosclerosis can be used to define risk factors, and used to prognosticate the outcome of vessel transplantation by estimating the probability of graft failure, which will aid physicians in determining the best course of treatment. The cellular and humoral components of blood removed from the site of the vessel transplantation that play a role in the mechanism of thrombosis can also be subject to microarray analysis.

It should be noted that gene expression levels may frequently correlate to observable, clinical events. Gene expression levels may serve to anticipate clinical events, such as improvements in clinical symptoms, and may also serve to confirm and refine clinical observations.

### EXAMPLES

### Example 1

### Materials and Methods of the Present Invention

In Vivo Sample Collection: Normal saphenous veins (“SPV”), radial artery (“RA”) and internal mammary artery (“IMA”) were obtained from the remnant of pre-implanted vessels. Aortic tissues were obtained from punched tissues during bypass surgery. Degenerated vein grafts were removed from failed hearts explanted from heart transplant patients. SPV, RA, and IMA samples were compared. Gene expression patterns among artery and vein samples that were tested were significantly different. Therefore, the selection of vessel type may affect the clinical outcome of whether vascular graft disease develops or not. Compared to IMA, SPV demonstrates lower long-term patency rate and accelerated arteriosclerosis. When the normal anatomical ambiance vein is compared to IMA, the anatomical ambiance vein does not correlate with arteriosclerosis. These provide examples of differential expression of genes in veins verses arteries, suggesting different clinical phenotypes for various veins and arteries. In addition, normal SPV and degenerated, or diseased, vein grafts removed from an explanted failed heart were compared, in order to identify genes that are activated or inhibited during various pathological processes. Identified genes can be incorporated into the various embodiments of the present invention.

In Vitro Cell Culture: Human coronary artery endothelial cells (“CAEC”), human saphenous vein endothelial cells (“SPVEC”), human coronary artery smooth muscle cells (“CASMC”), and human saphenous vein smooth muscle cells (“SPVSMC”) were primary cultured cells obtained from BioWhitakker, Inc. Cells were employed for studies at passage 6. All cell types were plated on 100-mm or 150-mm culture dishes pre-coated with 2% gelatin (Sigma, St. Louis, Mo.), and cultured in a defined cell culture medium from BioWhitakker Inc. After culturing in a serum-free medium for 12 hrs., cells at approximately 80% confluence were treated with different growth factors (TNα, TGFβ, IL1β, Oxidized-LDL and PDGF) for over night. Gene expression patterns for endothelial (“EC”) and smooth muscle cells (“SMC”) isolated from veins and arteries, in basal states and treated with different growth factors, were compared. Results from the in vitro study may be used to identify specific cell types that contribute to the in vivo results observed. Venous and arterial cells in culture were compared with respect to various types of treatment with growth factors, such as TNFα and IL1β, which are inflammatory cytokines that play important roles in arteriosclerosis. PDGF, which increases SMC proliferation and migration, was also used. TGFβ, which inhibits SMC proliferation and migration, was also used. OX-LDL stimulates SMC and invokes inflammatory responses of vascular wall. These results demonstrate that veins and arteries respond differently in response to growth factors, which may be one consideration of many, for selecting graft candidates. Identified genes can be incorporated into the various embodiments of the present invention.

RNA Isolation: The tissues for RNA extraction were immediately treated in RNALater reagent (Qiagen, Valencia, Calif.) and stored in −20° C. for 2-4 days before RNA extraction. Tissues were quickly frozen in liquid nitrogen, and tissues were reduced to powder form using a mechanical device. The powdered tissue sample was homogenized with Handishear (Virtis) in TRIZOL reagent (Invitrogen, Carlsbad, Calif.). Parts of fresh tissues were fixed in formalin for histological study. For cultured vascular cells, the cells were harvested in Trizol reagent and stored at −80° C. until RNA extraction. The aqueous layer after phenol-chloroform extraction was applied to the RNeasy column (Qiagen, Valencia, Calif.) for further purification. RNA concentration was quantified by a Nanodrop (National Instruments) and by UV spectrometer. The integrity of all samples was checked using BioAnalyzer 2100 (Agilent, Palo Alto, Calif.). http://www.labs.agilent.com/resources/techreports.html

Total RNA Labeling and Hybridization for Agilent Oligo Microarray: Total RNA from samples or common reference RNA (Universal Mouse RNA, Stratagene, La Jolla, Calif.) was labeled with Low RNA Input Fluorescent Linear Amplification kit (Agilent, Palo Alto, Calif.). Briefly, 500 ng of total RNA was primed with T7 promoter primer and reversed transcribed at 40° C. for 2 hrs. in the presence of 400 units of MMLV-RT, 50 μM each dATP, dTTP, dGTP and cCTP; and 1 units of RNaseOUT. Fluorescent cRNA was synthesized by in vitro transcription and by incorporation of cyanine 3-CTP or cyanine 5-CTP. The incubation was carried out at 40° C. for 2 h in the presence of T7 polymerase, NTP mix, PEG, inorganic phosphate and RNaseOUT. The labeled cRNA and amount of cyanine-dye incorporation were quantified by a Nanodrop (National Instruments). 1 μg each of cyanine 5- and cyanine 3-labeled cRNA were mixed together and hybridized on a Custom Human Oligonucleotide Microarray for 17 hrs. at 60° C. in a rotating oven. The Custom Human Oligonucleotide Microarray contains approximately 21,600 unique transcripts or genes covering all known cardiovascular genes. http://www.labs.agilent.com/resources/techreports.html.

Alternatively, Total RNA or mRNA can be labeled with can be labeled with Agilent Direct Labeling Kit (Agilent, Palo Alto, Calif.). Briefly, 10 μg of total RNA from tissue samples or common reference RNA (Universal Pooled Human Reference RNA, Stratagene, La Jolla, Calif.) were primed with oligo d(T) and reverse transcribed in the presence of 400 units of Superscript II RNase H Reverse Transcriptase (Invitrogen, Carlsbad, Calif.). The labeling reaction was incubated at 42° C. for 1 hour in the presence of 100 μM each dATP, dTTP, and dGTP; 25 μM dCTP; 25 μM of Cy3- or Cy5-dCTP (NEN Life Science, Boston, Mass.); and 27 units of RNAguard Ribonuclease Inhibitor (Amersham, Piscataway, N.J.), followed by RNase I degradation of unlabeled RNA. Labeled cDNAs were purified with the Qiaquick PCR cleanup kit (Qiagen, Valencia, Calif.). All purified targets were hybridized on Agilent oligo microarray.

Scanning, Background Subtraction and Normalization of Microarray Data: The slides were washed and scanned on an Agilent G2565AA Microarray Scanner System. Images were quantified using Agilent Feature Extraction Software (Version A. 7.1.2). Processing included local background subtraction with global adjustment and rank consistency based on a probe selection filter. Normalization was carried out using a LOWESS algorithm. Resolver Software was used to perform data analysis, and Rank Consistency Score was applied to determine the significant cutoff of gene expression levels as described (Chen et al., Circulation 108: 65-72, 2003). Microarrays were scanned on an Agilent G2565AA Microarray Scanner System. Images were quantified using Agilent Feature Extraction Software (Version A.6.1.1). Processing included local background subtraction and a rank consistency based probe selection filter. Normalization was carried out using a LOWESS algorithm 25. Dye-normalized signals of cy3 and cy5 channels were used in calculating log ratios. Ratios were averaged for each dye swap using the arithmetic mean.

Analysis of Microarray Data: Several statistical methods could be used to identify those genes that are likely to be differentially expressed between two or more different tissue types. These methods fall into two broad categories: parametric and non-parametric (distribution free) scoring methods. Parametric methods assume a certain distribution for expression values of every gene within each given class (e.g., tissue type) and then score genes according to how separate the class specific distributions are. Examples of the parametric method are the t-test (Rice, J. A., Mathematical Statistics and Data Analysis, Duxbury Press, 1995), and the Gaussian Error score (Ho M., Yang E., Matcuk G., Deng D., Sampas N., Tsalenko A., Tabibiazar R., Zhang Y., Chen M., Talbi S., Ho Y. D., Wang J., Tsao P. S., Ben-Dor A., Yakhini Z., Bruhn L., Quertermous T., Identification of endothelial cell genes by combined database mining and microarray analysis, Physiol Genomics. 2003 May 13;13(3):249-62). Distribution free scores, in contrast, are not based on parametric assumptions. Examples of such scores include the Kolmogorov-Smirnov score, the Wilcoxon rank-sum test (Chakravarti, L. and J. Roy. 1967. Handbook of Methods of Applied Statistics, Volume 1. John Wiley & Sons, New York, N.Y.; Hollander, M. and D. A. Wolfe. 1973. Nonparametric Statistical Methods. John Wiley & Sons, New York, N.Y.), TNoM score (A. Ben-Dor, L. Bruhn, N. Friedman, I. Nachman, M. Schummer, and Z. Yakhini, Tissue classification with gene expression profiles. J Comput Biol 7: 559-583, 2000; A. Ben-Dor, N. Friedman, and Z. Yakhini, Class discovery in gene expression data, In Proceedings of the Fifth International Conference on Computational Biology, pp. 31-38, 2001), and Rank consistency score (M. M. Chen, E. A. Ashley, D. X. Deng, A. Tsalenko, A. Deng, R. Tabibiazar, A. Ben-Dor, B. Fenster, E. Yang, J. Y. King, M. Fowler, R. Robbins, F. L. Johnson, L. Bruhn, T, McDonagh, H. Dargie, Z. Yakhini, P S Tsao, T Quertermous. Novel role for the potent endogenous inotrope apelin in human cardiac dysfunction, Circulation 108: 65-72, 2003). The use of non-parametric scores for microarray data analysis have been described previously (A. Ben-Dor, L. Bruhn, N. Friedman, I. Nachman, M. Schummer, and Z. Yakhini, Tissue classification with gene expression profiles. J Comput Biol 7: 559-583, 2000; A. Ben-Dor, N. Friedman, and Z. Yakhini, Class discovery in gene expression data, In Proceedings of the Fifth International Conference on Computational Biology, pp. 31-38, 2001). Non-parametric scores tend to be not as sensitive to the effects of outliers and are not based on the assumption of homogenous distribution within each class of samples. Gaussian error score can be used to find genes differentially expressed between more than two classes of samples. For each of the scoring methods mentioned above, the corresponding p-values can be computed exactly (e.g. for TNoM score) or approximated using the permutation test or corresponding distributions (e.g. for t-test). These p-values could be used to determine the overabundance of genes differentially expressed between two classes of samples (e.g. between arterial and saphenous vein samples) at any significance level using binomial surprise rate (A. Ben-Dor, L. Bruhn, N. Friedman, I. Nachman, M. Schummer, and Z. Yakhini, Tissue classification with gene expression profiles. J Comput Biol 7: 559-583, 2000), or false discovery rate (FDR) (Tusher, V. G., R. Tibshirani, and G. Chu., Significance analysis of microarrays applied to the ionizing radiation response. Proc Natl Acad Sci U S A 98: 5116-5121, 2001).

In Vitro comparison of smooth muscle cells in coronary artery and saphenous vein. For the 320 genes identified, the respective t-test p-value representing the comparison of saphenous vein smooth muscle cells and coronary artery smooth muscle cells was less than 0.001. Under random model, only 17 such genes in our microarray data were expected, which corresponds to the false discovery rate of 0.05.

In Vivo comparison of saphenous vein samples vs. RA and IMA samples. For the 232 genes identified, the respective t-test p-value for the comparison of saphenous vein samples and RA and IMA samples was less than 0.001. Under random model, only 21 such genes in our microarray data were expected, which corresponds to the false discovery rate of 0.09.

Microarrays: Two Agilent in situ oligo microarrays were utilized. One is commercial available as “ADHOC1A array” containing about 17,300 validated unique genes or transcripts for in vitro cell culture studies. Another array is a custom oligo array that covers almost all known genes related to cardiovascular system in addition to all genes from the ADHOC1A array, with 21,600 unique genes or transcripts. The custom oligo array was used for the analysis of in vivo samples.

The foregoing description, for purposes of explanation, used specific nomenclature to provide a thorough understanding of the invention. However, it will be apparent to one skilled in the art that the specific details are not required in order to practice the invention. The foregoing descriptions of specific embodiments of the present invention are presented for purpose of illustration and description. They are not intended to be exhaustive or to limit the invention to the precise forms disclosed. Obviously many modifications and variations are possible in view of the above teachings. The embodiments are shown and described in order to best explain the principles of the invention and its practical applications, to thereby enable others skilled in the art to best utilize the invention and various embodiments with various modifications as are suited to the particular use contemplated. It is intended that the scope of the invention be defined by the following claims and their equivalent.

### APPENDIX A

This Appendix section provides a list of the genes related to evaluation of candidate graft tissues grouped according to various known functions, such as cell proliferation and migration, inflammation and immune response, coagulation and thromobosis, extracellular matrix and cellular adhesion, transcription regulation, signal transduction, and other functions.

| | |
|---|---|
| GeneName | Description |

Cell proliferation and migration

| | | |
|---|---|---|
| | CDCA4 | cell division cycle associated 4 |
| NM_000076.1 | CDKN1C | Homo sapiens cyclin-dependent kinase inhibitor 1C (p57, Kip2) (CDKN1C), mRNA |
| I_934604 | BDNF | Brain-derived neurotrophic factor, a nerve growth factor that interacts with the Trk family of receptor |
| | | tyrosine kinases that stimulates differentiation of neurons and the growth of axons, regulates |
| | | synaptic transmission, may promote neuronal survival. Secreted by EC & protect SMC from |
| | | apoptosis. |
| I_930573 | IGFBP3 | Insulin-like growth factor binding protein 3, member of a family of proteins that binds to insulin-like |
| | | growth factors (IGF1, IGF2), inhibits IGF1 - induced proliferation and induces apoptosis in an IGF1 - |
| | | independent manner through interactions with PXRA |
| I_1002080 | IGFBP2 | Insulin like growth factor binding protein 2, binds to and modulates insulin-like growth factor activity, |
| | | regulates cell proliferation, may be involved in apoptosis; associated with the malignant phenotype, |
| | | may play a role in prostatic involution |
| I_961326 | IGFBP4 | Insulin-like growth factor binding protein 4, binds IGF1 and IGF2 to modulate cell growth, |
| | | proliferation, and probably differentiation, involved in developmental processes, implicated in |
| | | polycystic ovary syndrome, growth retardation and thyroid carcinoma |
| I_932459 | IGFBP6 | Insulin-like growth factor binding protein 6, member of the insulin-like growth factor binding protein |
| | | family, binds IGF2 with much greater affinity than IGF1 and inhibits IGF2-induced cell proliferation |
| I_956961 | IGF2R | Insulin-like growth factor II receptor, functions in transport of mannose 6-phosphate-containing |
| | | lysosomal enzymes and IGF-II maturation and clearance, mediates granzyme B-induced apoptosis, |
| | | putative tumor suppressor |
| I_958610 | TGFBI | Transforming growth factor beta induced, extracellular adhesion protein induced by transforming |
| | | growth factor beta (TGFB1) that may play roles in osteogenesis and lung structure/function; |
| | | alteration of corresponding gene causes several comeal dystrophies |
| D13628.2 | ANGPT1 | Angiopoietin 1, a ligand for the endothelial cell-specific receptor protein tyrosine kinase TIE2 (TEK), |
| | | stimulates cell migration and protects against apoptosis, plays a role in hemopoiesis, angiogenesis, |
| | | and utero-fetoplacental circulation |
| AK075219.1 | ANGPT2 | Angiopoietin 2, an antagonist and inhibitor of angiopoietin-1 (ANGPT1)-induced angiogenesis and |
| | | chemotaxis, acts through the endothelial protein tyrosine kinase receptor Tie2 (TEK), may promote |
| | | tumor cell survival and regulate tumor angiogenesis |
| BC011976.1 | CXCL1 | Growth related oncogene (melanoma growth stimulating activity), a CXC chemokine that binds |
| | | interleukin 8 receptor to mobilize intracellular calcium, acts as a leukocyte mitogenic factor with |
| | | growth-regulatory and chemotactic properties during . . . |
| NM_000609.1 | CXCL12 | Stromal cell-derived factor 1, an alpha chemokine that acts through the G protein-coupled receptor |
| | | CXCR4, stimulates leukocyte adhesion, migration and chemotaxis, inhibits infection by lymphocyte- |
| | | tropic HIV-1 strains |
| BC015753.1 | CXCL2 | Chemokine (C—X—C motif) ligand 2, a member of the C—X—C chemokine family, acts as a |
| | | neutrophil chemoattractant and epithelial cell mitogen, involved in immune and inflammatory |
| | | responses and may modulate glutamatergic synaptic transmission |
| BC016308.1 | CXCL3 | Melanoma growth stimulating activity gamma, a chemokine and mitogenic factor, activates |
| | | neutrophils and induces chemotaxis, may be involved in the inflammatory response; increased |
| | | expression may correlate with inflammation in hemolytic-uremic syndrome |
| U81234.1 | CXCL6 | Small inducible cytokine subfamily B member 6 (granulocyte chemotactic protein 2), ligand for |
| | | CXCR1 and CXCR2, promotes neutrophilic granulocyte chemotaxis and gelatinase B (MMP9) |
| | | release and may play a role in inflammation |
| I_1109403 | LTBP1 | Latent transforming growth factor beta binding protein 1, plays a role in latent TGF-beta 1 (TGFB1) |
| | | assembly, secretion, targeting to the extracellular matrix; variants may be associated with coronary |
| | | heart disease, marker for PEX syndrome |
| AB037158.1 | DSCR6 | Down syndrome critical region gene 6, contains a Leishmanolysin domain, may function in early |
| | | embryogenesis; corresponding gene is located in the Down syndrome critical region of |
| | | chromosome 21, suggesting a possible role in Down syndrome pathogenesis |
| BC030828.1 | EDIL3 | EGF-like repeats and discoidin I-like domains 3, may be a ligand for the alpha5beta3 integrin |
| | | receptor, may promote endothelial cell attachment and migration, may be involved in vascular |
| | | morphogenesis or remodeling |
| M60828.1 | FGF7 | Fibroblast growth factor 7 (keratinocyte growth factor), stimulates epidermal cell proliferation and |
| | | differentiation during wound healing and lung development, plays a role in cell cycle, apoptosis, |
| | | angiogenesis, and cell migration |
| BC018650.1 | EDG1 | Endothelial differentiation gene-1, a G protein-coupled receptor activated by sphingosine-1- |
| | | phosphate, signals through Gi proteins and Rac, Ras and Rho signaling pathways, promotes cell |
| | | adhesion, migration, and angiogenesis, inhibits apoptosis |
| BC036034.1 | EDG2 | Endothelial differentiation lysophosphatidic acid G protein-coupled receptor 2, a lysophosphatidic |
| | | acid receptor, involved in apoptosis and cell proliferation, may be a negative regulator for ovarian |
| | | epithelial cell growth and metastasis |
| D50405.1 | HDAC1* | Histone deacetylase 1, protein deacetylase and transcriptional corepressor, controls cell |
| | | proliferation through deacetylation of histones, p53 (TP53), and YY1, and through interactions with |
| | | pRb (RB1), topoisomerase II beta (TOP2B), and DNMT1 |
| AK096149.1 | MIG-6* | Mitogen-inducible gene 6, putative tumor suppressor expressed in a cell cycle dependent manner, |
| | | activates c jun N terminal kinase (human MAPK10), may mediate ERBB2 signaling |
| BC017197.1 | MCL1 | Myeloid cell leukemia 1, an apoptosis inhibitor sharing sequence homology with Bcl-2 (BCL2), is |
| | | upregulated by survival promoting ligands during cell differentiation; elevated expression is |
| | | associated with poor prognosis of oligodendroglioma patients |
| BC018695.1 | MEST | Mesoderm specific protein, a paternally expressed member of the alpha-beta hydrolase fold family, |
| | | functions in mesoderm development and may play a role in angiogenesis; loss of gene imprinting is |
| | | linked with lung and breast carcinomas |
| NM_003243.1 | TGFBR3 | Transforming growth factor beta receptor type III (betaglycan), presents TGFbeta to its signaling |
| | | receptor which enhances the cellular response to TGFbeta, may be involved in development; |
| | | increased expression may be therapeutic for breast cancer |
| U27185.1 | RARRES1 | Retinoic acid receptor responder 1 (tazarotene-induced gene 1), a putative transmembrane protein, |
| | | may negatively regulate cell proliferation, expression is induced by the retinoic acid receptor- |
| | | specific retinoid tazarotene that is used to treat psoriasis |
| M76979.1 | SERPINF1 | Pigment epithelium-derived factor, a neuroprotective protein that inhibits angiogenesis in the eye, |
| | | regulates apoptosis and may play a role in cell differentiation, may be an antitumor agent in |
| | | neuroblastoma, may be used to treat retinal vasculopathies |
| NM_004426.1 | PHC1 | Early development regulator 1, may negatively regulate homeotic gene expression, may play roles |
| | | in cell proliferation, differentiation, and B-cell maturation; may be involved in CATCH22 syndrome |
| | | and childhood B-cell precursor acute lymphoblastic leukemia |

Inflammation and Immune Response

| | | |
|---|---|---|
| I_929482 | IL6 | Interleukin 6 (interferon-beta 2), cytokine that promotes the proliferation of T and B cells and final |
| | | maturation of B-cells; implicated in multiple sclerosis and the persistence of multiple myeloma and |
| | | B-chronic lympohcytic leukemia |
| I_942167 | IL1B | Interleukin 1 beta, cytokine that modulates defense and inflammatory responses, plays a central |
| | | role in leukemic arthritis, may also contribute to autoimmune diabetes and pancreatic cancer |
| | | metastasis |
| I_957620 | IL8 | Interleukin 8, a cytokine that plays a role in chemoattraction and activation of neutrophils, involved |
| | | in immune and inflammatory responses |
| I_957623 | GRO1 | Growth related oncogene (melanoma growth stimulating activity), a CXC chemokine that binds |
| | | interleukin 8 receptor to mobilize intracellular calcium, acts as a leukocyte mitogenic factor with |
| | | growth-regulatory and chemotactic properties during inflammation |
| I_957614 | GRO2 | Macrophage inflammatory protein 2, a member of the C—X—C chemokine family, acts as a neutrophil |
| | | chemoattractant and epithelial cell mitogen |
| I_957616 | GRO3 | Melanoma growth stimulating activity gamma, a chemokine and mitogenic factor, activates |
| | | neutrophils and induces chemotaxis, may be involved in the inflammatory response |
| I_930918 | IL18BP | Interleukin 18 binding protein, immunoglobulin superfamily member that binds to and antagonizes |
| | | interleukin-18 (IL18), provides feedback inhibition of IL18-induced interferon-gamma (IFNG) |
| | | production, functions in wounding and inflammatory responses |
| I_928524 | TNFRSF14 | Tumor necrosis factor receptor superfamily member 14, part of a signal transduction pathway that |
| | | activates transcription factors AP-1 and NF-kappaB, mediates entry of herpes simplex virus |
| I_931262 | TNFSF8 | Tumor necrosis factor (ligand) superfamily, member 8, type II transmembrane protein that binds |
| | | CD30 receptor (TNFRSF8) and regulates the proliferation and differentiation of lymphocytic and |
| | | myeloid cells; involved in the pathogenesis of Hodgkins disease |
| I_928321 | VCAM1 | Vascular cell adhesion molecule 1, an immunoglobulin superfamily member that mediates |
| | | recruitment and adhesion of specific leukocytes to endothelial cells during the inflammatory |
| | | response and may have a role in arteriosclerosis |
| I_958485 | ESM1 | Endothelial cell-specific molecule 1, a cysteine-rich secreted protein, may participate in vascular |
| | | endothelial function and play a role in local inflammation by modulating endothelial cell-leukocyte |
| | | interactions |
| I_959599 | IFI27 | Interferon alpha-inducible protein 27, a putative membrane protein that is induced by interferon |
| | | alpha expressed in activated dendritic cells and overexpressed in breast carcinomas |
| I_932002 | THY1 | Thymus cell surface antigen, a GPI anchored protein and member of the immunoglobulin |
| | | superfamily, expressed on brain tissue, thymocytes, and various cells of the hematopoietic lineage |
| Y08768.1 | IL13RA2 | Interleukin 13 receptor alpha 2, a high affinity interleukin-13 (IL13) receptor that is also involved in |
| | | the intemalization of IL13, may play a role in the inflammatory response |
| I_962330 | MX1 | Myxovirus influenza resistance 1, a GTPase that is a member of the dynamin large GTPase |
| | | superfamily; is induced by interferon and is involved in antiviral defense against RNA viruses; |
| | | downstream target of Fanconi anemia protein FAC (FANCC) |
| I_962329 | MX2 | Myxovirus (influenza virus) resistance 2 (mouse), member of the GTPase superfamily, has a |
| | | proline-rich domain and a leucine zipper, a GTPase that forms oligomers; corresponding gene is |
| | | overexpressed in a prostate cancer cell line |
| NM_001928.1 | DF | Adipsin (complement factor D), a serine protease that functions in the alternative complement |
| | | pathway to cleave complement factor B when complexed to component C3, deficiency in humans |
| | | results in increased susceptibility to Neisseria meningitidis |
| M33552.1 | LSP1 | Lymphocyte-specific protein 1, leukocyte-specific protein that binds F-actin, acts in differentiation |
| | | and signal transduction, negatively regulates neutrophil migration; overexpression is associated |
| | | with neutrophil actin dysfunction (NAD 47/89) |
| NM_006905.2 | PSG1 | Pregnancy specific beta-1-glycoprotein 1, a member of the immunoglobulin superfamily, induces |
| | | the secretion of anti-inflammatory cytokines by monocytes and may modulate the immune system |
| M34420.1 | PSG3 | Pregnancy specific beta-1-glycoprotein 3, member of the pregnancy-specific glycoprotein (PSG) |
| | | subgroup of the carcinoembryonic antigen (CEA) family |
| M33666.1 | PSG6 | Pregnancy specific glycoprotein 6, member of carcinoembryonic antigen family which is a subfamily |
| | | of the immunoglobulin superfamily, expressed predominantly in placenta |
| U18467.1 | PSG7 | Pregnancy-specific glycoprotein 7, member of the carcinoembryonic antigen family, expressed in |
| | | the liver and placenta |
| I_959180 | SCYA2 | Cytokine A2, CC chemokine that attracts monocytes, memory T-cells, natural killer cells and |
| | | endothelial cells, plays a role in the inflammatory response to infection and in inflammatory |
| | | diseases including arthritis, multiple sclerosis and arteriosclerosis |
| I_959599 | IFI27 | Interferon alpha-inducible protein 27, a putative membrane protein that is induced by interferon |
| | | alpha expressed in activated dendritic cells and overexpressed in breast carcinomas |

Coagulation and Thrombogensis

| | | |
|---|---|---|
| I_961844 | THBD | Thrombomodulin, a plasma membrane receptor that negatively regulates blood coagulation; |
| | | mutation of the corresponding gene results in an increased risk of myocardial infarction |
| U59632.1 | GP1BB | Glycoprotein Ib (platelet) beta polypeptide, component of the von Willebrand factor receptor that |
| | | mediates platelet activation; mutation of the corresponding gene is associated with Bernard Soulier |
| | | syndrome and hereditary giant platelet disorder |
| BC007231.1 | PLAT | Tissue-type plasminogen activator, a serine protease that converts inactive plasminogen to |
| | | plasmin, functions in fibrinolysis; genetic deficiencies are associated with increased risk for |
| | | thrombosis |
| BC011171.1 | SERPING1 | Serine (or cysteine) proteinase inhibitor, regulates the classical complement pathway through |
| | | inhibition of C1, inhibits the alternative complement pathway, blood coagulation and vascular |
| | | permeability; mutations causes hereditary angioedema |
| NM_006216.1 | SERPINE2 | Serine (or cysteine) proteinase inhibitor clade E (nexin, plasminogenactivator inhibitor type 1) |
| | | member 2, forms complexes with urokinase, plasmin, and thrombin; may be involved in the |
| | | pathogenesis of Alzheimer disease and scleroderma |

Extracellular Matrix and Cellular Adhesion

| | | |
|---|---|---|
| I_930282 | MMP10 | Matrix metalloproteinase 10 (stromelysin 2), catalyzes the activation of other matrix |
| | | metalloproteases, involved in degradation of the extracellular matrix, may play a role in tumor cell |
| | | invasion; mRNA is overexpressed in diabetic retinopathy comeas |
| I_962112 | TIMP1 | Tissue inhibitor of metalloproteinase 1, inhibits matrix metalloproteases, forms complex with MMP9 |
| | | at the cell surface, and contributes to cell proliferation and stress responses, attenuates metastasis |
| | | of tumorigenic cells when overexpressed |
| I_960905 | TIMP2 | Tissue inhibitor of metalloproteinase 2, a metalloprotease inhibitor Which inhibits gelatinase A |
| | | (MMP2), functions in cell proliferation, antiapoptosis, and inhibition of angiogenesis, may inhibit |
| | | tumor metastasis |
| I_958653 | THBS1 | Thrombospondin-1, an extracellular matrix glycoprotein that interacts with other matrix proteins and |
| | | cell surface receptors to regulate cell attachment, spreading, migration, and proliferation; an |
| | | inhibitor of angiogenesis and tumor growth |
| I_964991 | THBS2 | Thrombospondin 2, an adhesive molecule that binds heparin and plays roles in cell adhesion and |
| | | inhibition of angiogenesis, may have roles in development and response to viruses; lack of |
| | | expression is important in the enhancement of angiogenesis in glioma |
| NM_030820.2 | COL21A1 | Protein containing six collagen triple helix repeats, von Willebrand factor (vWF) type A and |
| | | thrombospondin N-terminal-like domains, has moderate similarity to collagen type IX alpha 1 |
| | | (human COL9A1), which is associated with multiple epiphyseal . . . |
| AB038518.1 | COLEC12 | Collectin subfamily member 12, a type II transmembrane glycoprotein that binds bacteria through |
| | | its lectin domain and may play a role in host defense, also binds LDL and may play a role in |
| | | atherogenesis |
| BC005322.1 | DCN | Decorin, a dermatan/chondroitin sulfate proteoglycan that binds to collagen and transforming |
| | | growth factor beta, negatively controls cell growth and may have a role during organogenesis, |
| | | deficiency is associated with Marfan syndrome |
| X74764.1 | DDR2 | Discoidin domain receptor 2, receptor protein tyrosine kinase that is activated by collagen, contains |
| | | discoidin motifs in the extracellular domain, upregulates matrix metalloproteinase 1, may be |
| | | involved in cell adhesion |
| BC037273.1 | OGN | Osteoglycin, a member of the keratan sulfate proteoglycan group of the small leucine-rich |
| | | proteoglycan family, may play a role in regulating comeal transparency |
| AF126110.1 | FBLN1 | Fibulin 1, an extracellular matrix glycoprotein that may connect extracellular matrix elements, may |
| | | play roles in hemostasis and limb development, tumor invasion, thrombosis, and connective tissue |
| | | and blood diseases |
| AF101051.1 | CLDN1 | Claudin-1, a member of the claudin family, contains four transmembrane domains, localizes to tight |
| | | junction strands, may maintain cell polarity and contribute to cell-cell adhesion and tight junction |
| | | formation, upregulated in colorectal tumors |
| BC010514.1 | CLU | Clusterin (apolipoprotein J), a component of a subclass of high density lipoproteins, binds bacteria, |
| | | involved in apoptosis, cell adhesion, and stress response, increased expression is associated with |
| | | various cancers and neurological diseases |
| M26326.1 | KRT18 | Keratin 18 (cytokeratin 18), a type I epidermal keratin and component of intermediate filaments, |
| | | may function in apoptosis, cell migration, and response to viruses, stress, and drug sensitivity; |
| | | mutation of the gene may cause cryptogenic cirrhosis |
| AK092499.1 | TFPI2 | Tissue factor pathway inhibitor 2, a member of the Kunitz-type serine protease inhibitor family that |
| | | is associated with the extracellular matrix, may play roles in maintaining placental tissue and |
| | | extracellular matrix integrity, and in tumor invasion |
| NM_002426.1 | MMP12 | Homo sapiens matrix metalloproteinase 12 (macrophage elastase) (MMP12), mRNA |
| U89942.1 | LOXL2 | Lysyl oxidase-like 2, scavenger receptor cysteine-rich family member, predicted to function in |
| | | cellular senescence, cell adhesion, and play a role in the development of the placenta and early |
| | | fetal membranes, upregulated in Werner syndrome fibroblasts |
| NM_006475 | OSF-2 | Osteoblast specific factor 2, a putative cell adhesion molecule that may play a role in homophilic |
| | | cell adhesion during bone formation, altered expression may contribute to the development of |
| | | melorheostosis, a rare bone disease |
| AF098269.1 | PCOLCE2 | Procollagen C-endopeptidase enhancer 2, a glycoprotein that enhances cleavage of type II |
| | | procollagen by the procollagen C-proteinases, BMP1 and TLL1; may be associated with open- |
| | | angle glaucoma |
| U70136.1 | PRG4 | Proteoglycan 4 (megakaryocyte stimulating factor), a secreted proteoglycan with possible roles in |
| | | regulating cell proliferation, ossification and joint lubrication; mutations in the gene cause |
| | | camptodactyly-arthropathy-coxa vara-pericarditis syndrome |
| NM_020404.1 | TEM1 | Tumor endothelial marker 1 precursor (endosialin), contains C-type lectin, Sushi/SCR/CCP and |
| | | EGF-like domains, cell surface glycoprotein on tumor blood vessel endothelium, may function as an |
| | | endothelial receptor and play a role in angiogenesis |
| NM_153370.1 | MGC45378 | Member of the SCP-like extracellular protein family, has a region of moderate similarity to a region |
| | | of protease inhibitor 15 (human PI15), which is a trypsin inhibitor involved in extracellular matrix |
| | | proteolysis and associated with tumor invasion |
| I_957267 | COL12A1 | Collagen type XII alpha 1, member of the family of fibril-associated collagens that have interrupted |
| | | triple helices (FACIT), may be involved in skeletal development, has reduced expression in |
| | | keratoconus comeas |
| I_932752 | COL13A1 | Alpha 1 subunit of type XIII collagen, a plasma membrane protein, may play a role in cell adhesion, |
| | | eye development, and eye function |
| I_929022 | COL16A1 | Collagen type XVI (alpha 1 subunit), component of extracellular matrix which is regulated by |
| | | TGFB1, probably involved in chondrogenesis and may be important in pregnancy |
| I_930569 | COL1A2 | Alpha 2 subunit of type I collagen, involved in cell-matrix adhesion and wound healing; mutations of |
| | | the gene are associated with Ehlers-Danlos syndrome type VII, systemic sclerosis and |
| | | osteogenesis imperfecta |
| I_928318 | COL3A1 | Collagen type III alpha 1, an extracellular matrix protein; mutation of corresponding gene can cause |
| | | Ehlers-Danlos syndrome type IV and familial aortic aneurysms |
| I_1100034 | COL6A1 | Collagen (type VI, alpha 1), may be involved in maintaining muscle fiber integrity, may contribute to |
| | | cell-matrix adhesion; mutations of the corresponding gene cause Bethlem myopathy and may be |
| | | associated with congenital heart defects in Down Syndrome |
| I_962369 | COL6A2 | Collagen (type VI, alpha 2), an extracellular matrix structural protein which may be involved in bone |
| | | mineralization, muscle development and wound healing; mutations in the corresponding gene |
| | | cause Bethlem myopathy and Ullrich syndrome |

Transcription Factors

| | | |
|---|---|---|
| X98054.1 | CREBL1 | AUTO: Creb (cyclic AMP-response element binding protein) related protein, a member of the |
| | | CREB/ATF family of transcription factors, a regulator . . . |
| M34309.1 | ERBB3 | Homo sapiens v-erb-b2 erythroblastic leukemia viral oncogene homolog 3 (avian) (ERBB3), mRNA |
| NM_004364.2 | CEBPA | CCAAT/enhancer binding protein alpha, transcriptional activator involved in adipogenesis and |
| | | granulocyte differentiation; gene mutations are associated with acute myeloid leukemia and |
| | | expression is decreased in lung and hepatocellular carcinomas |
| AY014299.1 | HOXC8 | Homeobox protein C8, a putative transcriptional repressor of the Hox protein family, interacts with |
| | | SMAD1 and plays a role in the regulation of cartilage differentiation; may be involved in the |
| | | tumorigenesis of cervical cancers |
| AF452494.1 | CEGF3* | Protein with high similarity to human SCUBE1, which is expressed in endothelial cells and highly |
| | | vascularized tissues and downregulated in response to proinflammatory cytokines, contains six |
| | | epidermal growth factor (EGF)-like domains and a CUB domain |
| AF140360.1 | HBOA | Histone acetyltransferase, member of the MYST family, contains a C2HC zinc finger, binds to |
| | | MCM2 and to ORC1L of origin recognition complex required for initiation of DNA replication, binds |
| | | to androgen receptor (AR) and acts as a transcriptional . . . |

Signal Transduction

| | | |
|---|---|---|
| NM_139047.1 | MAPK8* | Mitogen activated protein kinase 8, a serine-threonine kinase that regulates c-Jun (JUN), acts in |
| | | receptor signaling, cell growth and differentiation, apoptosis, and response to stressors such as |
| | | DNA damage, reactive oxygen, hypoxia and rRNA damage |
| AF432215.1 | NCOA6IP | nuclear receptor coactivator 6 interacting protein |
| NM_005019.2 | PDE1A | Calmodulin-dependent phosphodiesterase 1A, a cyclic nucleotide phosphodiesterase that has |
| | | higher affinity for cGMP than cAMP, may play a role in penile erection |
| BC000737.1 | RGS4 | Regulator of G protein signaling 4, acts as a GTPase-activating protein (GAP) towards Galpha-i |
| | | and Galpha-q subunits to attenuate G protein signaling activity, expression is elevated in failing |
| | | hearts and decreased in the cortex of schizophrenics |
| AB008109.1 | RGS5 | Regulator of G protein signaling 5, may bind to and act as a GTPase activating protein (GAP) for |
| | | Galpha subunits, negatively regulates G protein-coupled receptor signaling: mRNA abundance is |
| | | increased in renal cell carcinoma |
| AL832345.1 | PKCe* | Protein kinase C epsilon, a diacylglycerol-activated and phospholipid dependent isoform of protein |
| | | kinase C, involved in cell growth and apoptosis; gene mutation may be associated with thyroid |
| | | cancer |
| I_1100710 | SOCS5 | Suppressor of cytokines signaling 5 (cytokine inducible SH2 protein 6), contains SH2 domain and |
| | | SOCS box, negatively regulates cytokine signaling via JAK-STAT pathways |
| BC030133.1 | SDC2 | Syndecan-2 (heparan sulfate proteoglycan 1), cell surface-associated, fibroglycan, acts as a CSF2 |
| | | coreceptor and may act in FGFR signaling cascade; plays a role in cell adhesion, cell proliferation, |
| | | and inflammatory response |
| M97675.1 | ROR1 | Neurotrophic tyrosine kinase receptor related 1, member of the ROR family of receptor tyrosine |
| | | kinases, may be involved in transmembrane receptor protein tyrosine kinase signaling pathways |
| BC005923.1 | MGST1 | Microsomal glutathione S-transferase 1, a glutathione transferase that is involved in the response |
| | | to oxidative stress |
| BC012423.1 | SOD2 | Superoxide dismutase 2, mitochondrial enzyme that catalyzes the conversion of superoxide to |
| | | hydrogen peroxide, plays a role in the response to oxidative stress, heat and radiation, helps |
| | | prevent apoptosis |
| NM_033546.1 | MLC-B | Protein with very strong similarity to myosin regulatory light chain (human MLCB), which is involved |
| | | in human RHO protein signal transduction and possibly the regulation of myosin head ATPase |
| | | activity in smooth muscle, contains three EF hand domains |

Others

| | | |
|---|---|---|
| NM_000625.3 | NOS2A* | Inducible nitric oxide synthase, calmodulin-binding enzyme that generates nitric oxide from arginine |
| | | and molecular oxygen, functions in antipathogen and inflammatory responses; upregulated by |
| | | interferon alpha and may protect against hepatitis C infection |
| L24470.1 | PTGFR* | Prostanoid FP receptor (prostaglandin F2-alpha receptor), activation induces calcium flux, |
| | | regulates smooth muscle contraction, and predicted to be necessary for luteolysis; mutations in the |
| | | corresponding gene are associated with breast cancer |
| Y00749.1 | EDN1* | Endothelin 1, a peptide hormone that acts through G protein-coupled receptor signaling, involved in |
| | | many physiologic processes including vasoconstriction and cell proliferation, upregulation is |
| | | associated with chronic heart failure |
| BC003672.1 | FABP4 | Fatty acid binding protein 4 (adipocyte fatty acid binding protein), binds retinoic acids, may have |
| | | role in fatty acid metabolism, upregulated by PPARG agonists; decreased expression is associated |
| | | with advanced stage bladder transitional cell carcinoma |
| BC016288.1 | FKBP3 | FK506-binding protein 3 (25 kDa), a rapamycin-selective DNA-binding nuclear immunophilin with |
| | | peptidylprolyl cis-trans-isomerase activity, associates with histone deacetylases HDAC1 and |
| | | HDAC2, transcription regulator YY1, and casein kinase II |
| NM_007270.1 | FKBP9 | Homo sapiens FK506 binding protein 9, 63 kDa (FKBP9), mRNA |
| I_962212 | ADH1A | Alcohol dehydrogenase 1A class I alpha polypeptide, a zinc-dependent alcohol, NAD+ |
| | | oxidoreductase that oxidizes ethanol |
| I_931241 | FLJ20539 | Protein of unknown function, has low similarity to uncharacterized human KIAA1906 |
| I_958186 | I_958186 | Protein of unknown function, has strong similarity to uncharacterized mouse 1810027I20Rik |
| I_963838 | I_963838 | Member of the metallothionein family, which are cysteine-rich heavy metal binding proteins |
| I_963082 | FLJ23577 | Protein of unknown function |

We claim:

1. An array comprising:
a substrate;
a set of control probe molecules that do not correlate with vascular graft disease, each control probe molecule identified as being expressed in vascular tissues at approximately constant level of expression; and
a set of probe molecules that target vascular-graft-disease-related gene sequences that are identified to correlate with vascular graft disease.

2. The microarray of claim 1 wherein the set of probe molecules targets a subset of gene sequences related to a pathological state clinically recognized as early thrombosis.

3. The microarray of claim 1 wherein the set of probe molecules targets a subset of gene sequences related to a pathological state clinically recognized as intimal hyperplasia.

4. The microarray of claim 1 wherein the set of probe molecules targets a subset of gene sequences related to a pathological state clinically recognized as graft arteriosclerosis.

5. The microarray of claim 1 wherein a vascular tissue sample to which the array is exposed comprises various types of autogenous vascular tissue graft removed from a patient.

6. The microarray of claim 1 wherein a vascular tissue sample to which the array is exposed comprises various types of artery and venous tissues.

7. The microarray of claim 1 wherein probes of the set of probe molecules target vascular-graft-disease-related gene sequences selected from among:
angiopoietin 1 (ANGPT1), angiopoietin 2 (ANGPT2), brain-derived neurotrophic factor (BDNF), cell division cycle associated 4 (CDCA4), homo sapiens cyclin-dependent kinase inhibitor 1C (CDKN1C), homo sapiens CCAAT/enhancer binding protein (CEBPA), CEGF3, claudin-1 (CLDN1), clusterin (CLU), COL21A1, collectin subfamily member 12 (COLEC12), creb (cyclic AMP-response element binding protein) related protein (CREBL1), a CXC chemokine (CXCL1), stromal cell-derived factor 1 (CXCL12), chemokine ligand 2 (CXCL2), melanoma growth stimulating activity gamma (CXCL3), small inducible cytokine subfamily B member 6 (CXCL6), decorin (DCN), discoidin domain receptor 2 (DDR2), adipsin/serine protease (DF), down syndrome critical region gene 6 (DSCR6), endothelial differentiation gene-1 (EDG1), endothelial differentiation lysophosphatidic acid G protein-coupled receptor 2 (EDG2), EGF-like repeats and discoidin I-like domains 3 (EDIL3), endothelin 1 (EDN1), homo sapiens v-erb-b2 erythroblastic leukemia viral oncogene homolog 3 (ERBB3), fatty acid binding protein 4 (FABP4), fibulin 1 (FBLN1), fibroblast growth factor 7 (FGF7), FK506-binding protein 3 (FKBP3), homo sapiens FK506 binding protein 9 (FKBP9), growth differentiation factor 3 (GDF3), glycoprotein 1b beta polypeptide (GP1BB), GRO1, GRO2, histone acetyltransferase (HBOA), histone deacetylase 1 (HDAC1), homeobox protein C8 (HOXC8), insulin-like growth factor binding protein 2 (IGFBP2), insulin-like growth factor binding protein 3 (IGFBP3), insulin-like growth factor binding protein 6 (IGFBP6), insulin-like growth factor binding protein 7 (IGFBP7), interleukin 13 receptor alpha 2 (IL13RA2), interleukin 8 (IL8), keratin 18 (KRT18), LIM domain only 7 (LMO7), lysyl oxidase-like 2 (LOXL2), lymphocyte-specific protein 1 (LSP1), mitogen activated protein kinase 8 (MAPK8), myeloid cell leukemia 1 (MCL1), mesoderm specific protein (MEST), member of the SCP-like extracellular protein family (MGC45378), microsomal glutathione S-transferase 1 (MGST1), mitogen-inducible gene 6 (MIG-6), MLC-B, homo sapiens matrix metalloproteinase 12 (MMP12), nuclear receptor coactivator 6 (NCOA6IP), inducible nitric oxide synthase (NOS2A), osteoglycin (OGN), osteoblast specific factor 2 (OSF-2), procollagen C-endopeptidase enhancer 2 (PCOLCE2), calmodulin-dependent phosphodiesterase 1A (PDE1A), early development regulator 1 (PHC1), tissue-type plasminogen activator (PLAT), proteoglycan 4 (PRG4), protein kinase C epsilon (PRKCE), pregnancy specific beta-1-glycoprotein 1 (PSG1), pregnancy specific beta-1-glycoprotein 3 (PSG3), pregnancy specific beta-1-glycoprotein 6 (PSG6), pregnancy specific beta-1-glycoprotein 7 (PSG7). Prostanoid FP receptor (PTGFR), retinoic acid receptor responder 1 (RARRES1), regulator of G protein signaling 4 ( RGS4), regulator of G protein signaling 5 ( RGS5), neurotrophic tyrosine kinase receptor related 1 (ROR1), syndecan-2 (SDC2), serine (or cysteine) proteinase inhibitor clade E (SERPINE2), pigment epithelium-derived factor (SERPINF1), serine (or cysteine) proteinase inhibitor (SERPING1), suppressor of cytokines signaling 5 (SOCS5), superoxide dismutase 2 (SOD2), tumor endothelial marker 1 precursor (endosialin) (TEM1), tissue factor pathway inhibitor 2 (TFPI2), transforming growth factor beta receptor type III (betaglycan) (TGFBR3), thrombomodulin (THBD), tissue inhibitor of metalloproteinase 1 (TIMP1), tissue inhibitor of metalloproteinase 3 (TIMP3), and osteoprotegerin (TNFRSF11B).

8. A method for screening a vascular tissue among candidate vessels, the method comprising:
removing a vascular tissue sample from a patient;
exposing a microarray, comprising a set of control probe molecules that do not correlate with vascular graft disease, each control probe molecule identified as being expressed in vascular tissues at approximately constant level of expression, and a set of probe molecules that target vascular-graft-disease-related gene sequences that are identified to correlate with vascular graft disease, to a sample solution prepared from the vascular tissue sample;
quantitatively determining relative gene expression levels for a set of vascular-graft-disease-related genes based on data obtained from the microarray; and
comparing the relative gene expression levels with pre-determined standard to evaluate the probable viability of the vascular tissue as a graft.

9. The method of claim 8 wherein the probe molecules target a subset of gene sequences related to a pathological state clinically recognized as early thrombosis.

10. The method of claim 8 wherein the probe molecules target a subset of gene sequences related to a pathological state clinically recognized as intimal hyperplasia.

11. The method of claim 8 wherein the probe molecules target a subset of gene sequences related to a pathological state clinically recognized as graft arteriosclerosis.

12. The method of claim 8 wherein the vascular tissue sample comprises various types of autogenous vascular tissue graft removed from a patient.

13. The method of claim 12 wherein the autogenous vascular tissue graft includes artery and venous tissues.

14. The method of claim 8 further comprising:
exposing the vascular tissue to one of:
a growth factor;
a plasmid encoding a growth factor;
an anti-platelet agent; and
an anti-coagulent agent.

15. A method to monitor an implanted vascular tissue graft, the method comprising:
removing a sample from a vascular tissue graft;
exposing a microarray, comprising a set of control probe molecules that do not correlate with vascular graft disease, each control probe molecule identified as being expressed in vascular tissues at approximately constant level of expression, and a set of probe molecules that target vascular-graft-disease-related gene sequences that are identified to correlate with vascular graft disease, to a sample solution prepared from the autogenous vascular tissue graft;
quantitatively determining a relative gene expression levels for a set of vascular-graft-disease-related genes based on data obtained from the microarray; and
comparing the relative gene expression levels with pre-determined standard to evaluate the probable viability of the vascular tissue as a graft.

16. The method of claim 15 wherein the probe molecules target a subset of gene sequences:
related to a pathological state clinically recognized as early thrombosis.

17. The method of claim 15 wherein the probe molecules target a subset of gene sequences related to a pathological state clinically recognized as intimal hyperplasia.

18. The method of claim 15 wherein the probe molecules target a subset of gene sequences related to a pathological state clinically recognized as graft arteriosclerosis.

19. The method of claim 15 wherein the autogenous vascular tissue graft comprises various types of autogenous vascular tissue graft removed from a patient.

20. The method of claim 15 wherein the autogenous vascular tissue graft includes artery and venous tissues.
